(19) United States
(12) Patent Application Publication
(10) Pub. No.: US 2026/0041628 A1
(43) Pub. Date: Feb. 12, 2026

(54) MULTI-COMPONENT COMPOSITION AND USES THEREOF

(71) Applicant: EDWARDS LIFESCIENCES CORPORATION, Irvine, CA (US)

(72) Inventor: Bhumica A. Amin, Modesto, CA (US)

(21) Appl. No.: 19/361,718

(22) Filed: Oct. 17, 2025

Related U.S. Application Data
(63) Continuation of application No. PCT/US2024/021890, filed on Mar. 28, 2024.
(60) Provisional application No. 63/497,437, filed on Apr. 20, 2023.

(51) Int. Cl. A61K 9/00; A61K 47/34; A61N 1/362

(52) U.S. Cl. CPC A61K 9/0024; A61K 47/34; A61N 1/3629

(57) ABSTRACT

A composition, system, and method for use at a native valve inside a heart of a subject comprises a composition that comprises a mixture that includes a first component and a second component. The second component is encapsulated in capsules in a manner that prevents its interaction with the first component until desired. The capsules are rupturable to expose the second component to the first component responsively to application of energy to the composition. Once the capsules are exposed, the composition is configured to adhere to one or more leaflets of the native valve inside the heart.

### CROSS-REFERENCES TO RELATED APPLICATIONS

This application is a continuation of Patent Cooperation Treaty (PCT) application No. PCT/US2024/021890, filed on Mar. 28, 2024, which claims the benefit of U.S. Provisional Patent Application No. 63/497,437, filed Apr. 20, 2023, the entire disclosures of which are incorporated by reference for all purposes.

### BACKGROUND

Many surgical and treatment technologies require multi-part solutions or compositions, such as adhesives or foams. Typically, one would mix the components in a single delivery device, such as a syringe, prior to injection into a desired location. However, because mixing of the components may initiate setting of the mixture or composition, the procedure becomes complex and very time sensitive. As such, it is unsuitable for any type of treatment which requires extensive durations, such as transluminal treatments.

There is thus a need in the art for a composition able to form a multi-part adhesive or foam, in situ.

### SUMMARY

In some implementations, a mixture comprises a first component and a second component, at least one of the components sequestered within frangible capsules that prevent interaction between the components. In some such implementations, the frangible capsules are formed of a bi-layer of amphiphilic molecules, e.g., a lipid bilayer comprising phospholipids. In this way, the capsules may be similar to a liposome or a vesicle.

In some implementations, a de-encapsulation device is used to de-encapsulate the encapsulated component. For example, the de-encapsulation device can de-encapsulate the component by applying energy (e.g., heat, ultrasound energy, electromagnetic radiation and/or electrical current) and/or a chemical that disrupts (e.g., ruptures) the capsules. In some implementations, exposing the first and second components to each other by disrupting the capsules allows the components to interact such that the mixture forms an at least partially solid body, e.g., an adhesive body. Alternatively or in addition, a curing-energy applicator is used to cure the mixture into an at least partially solid form.

In some implementations, the mixture is disposed at a subject's anatomical site within a delivery structure, e.g., on a lattice structure holding the mixture. For some such applications, the delivery structure is delivered to the anatomical site with the mixture predisposed within the delivery structure. Alternatively or in addition, the mixture may be added to the delivery structure after the delivery structure is delivered to the anatomical site.

In some implementations, de-encapsulating the mixture's encapsulated component forms (e.g., shapes or creates) an implant. In some such implementations, the delivery structure may be bioabsorbed and/or removed from the subject, such that the mixture defines the implant. Alternatively, the delivery structure can remain at the anatomical site and define, together with the mixture, at least part of the implant. For example, composition 100 can increase the implant's stiffness, and/or form an adhesive that adheres the implant to the anatomical site and/or strengthens the anchoring of the implant to the anatomical site.

Any of the method(s) disclosed herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

There is therefore provided, in accordance with some implementations, a system for use at an anatomical site of a real or simulated subject, the system including an implant that includes: a porous matrix, and/or a composition, held in association with the matrix, the composition including a mixture that includes: a first component, and/or a second component, encapsulated in capsules in a manner that prevents its interaction with the first component, the capsules being rupturable to expose the second component to the first component responsively to application of energy to the composition.

For some implementations, the implant is sterile.

For some implementations, the capsules include polymer capsules.

For some implementations, the capsules include protein capsules.

For some implementations, the capsules include lipid bi-layer capsules.

For some implementations, the first component is a fluid medium, having suspended therein the capsules.

For some implementations, the mixture further includes a suspension medium in which the first component and the capsules are suspended.

For some implementations, the mixture includes a powder including the first component and the capsules.

For some implementations, the first component and the second component are two components of a two-part foam.

For some implementations, the first component and the second component are two components of a two-part polyurethane adhesive.

For some implementations, the first component and the second component are two components of a two-part resin.

For some implementations, the first component and the second component are two components of a two-part epoxy resin.

For some implementations, the mixture further includes a third component.

For some implementations, the system further includes: a delivery tool, configured to transluminally deliver the implant to the anatomical site, and/or a grasper, configured to hold the implant in contact with tissue at the anatomical site.

For some implementations, the system further includes a de-encapsulation device configured to: be transluminally advanced via the delivery tool to the anatomical site, and/or expose the second component to the first component by de-encapsulating the second component while the grasper holds the matrix in contact with the tissue at the anatomical site.

For some implementations: the grasper is configured to controllably release the matrix; and/or the composition is configured such that exposing the second component to the first component adheres the matrix to the tissue at the anatomical site, such that the matrix remains adhered to the tissue after the grasper releases the matrix.

For some implementations, the capsules are second capsules wherein: the first component is encapsulated in first capsules, and/or the de-encapsulation device is adapted to expose the first component and the second component to each other, by de-encapsulating the first component and the second component while grasper holds the matrix in contact with the tissue at the anatomical site.

For some implementations, the de-encapsulation device includes: a first de-encapsulation device adapted to expose the first component by de-encapsulating the first component from the first capsules; and/or a second de-encapsulation device adapted to expose the second component by de-encapsulating the second component from the second capsules, such that the first component and the second component are exposed to each other.

For some implementations, the mixture further includes a suspension medium in which both the first capsules and the second capsules are suspended.

For some implementations, the mixture includes a powder including the first capsules and the second capsules.

For some implementations, the mixture further includes a third component.

For some implementations, the third component includes a reinforcing component adapted for mechanically reinforcing a repair structure formed by the first component and the second component, subsequent to exposure of the second component by the de-encapsulation device.

For some implementations, the third component includes an indicator component adapted to enable visualizing of a location of the mixture within the subject, during or following delivery of the mixture by the delivery tool.

For some implementations, the third component includes a surfactant.

For some implementations, the third component includes a medicament.

For some implementations, the medicament is an anti-inflammatory medicament.

For some implementations, the medicament has tissue-growth-promoting properties.

For some implementations, the medicament has antimicrobial properties.

For some implementations, the de-encapsulation device includes a chemical adapted, upon introduction into the mixture, to carry out a chemical reaction resulting in de-encapsulating of the second component.

For some implementations, the de-encapsulation device includes an energy applicator adapted to apply energy to the mixture.

For some implementations, the energy applicator includes an electrode adapted to apply electrical energy to the mixture.

For some implementations, the energy applicator includes an electromagnetic radiation source adapted to apply electromagnetic energy to the mixture.

For some implementations, the energy applicator includes an ultrasound transducer.

For some implementations, the ultrasound transducer is adapted to apply high frequency ultrasound energy.

For some implementations, the ultrasound transducer is adapted to apply high-intensity focused ultrasound (HIFU) energy.

For some implementations, the ultrasound transducer is an intracorporeal ultrasound transducer.

For some implementations, the ultrasound transducer is an extracorporeal ultrasound transducer.

For some implementations, the first component of the mixture is also encapsulated, and the ultrasound transducer is adapted to apply ultrasound energy at a single frequency to de-encapsulate the first component and the second component.

For some implementations, the first component is also encapsulated, and the ultrasound transducer is adapted to apply ultrasound energy at a first frequency to de-encapsulate the first component and at a second frequency to de-encapsulate the second component.

For some implementations, a first subset of the capsules includes capsules having a first degree of strength, and a second subset of the capsules includes capsules having a second degree of strength, and/or the de-encapsulation device is adapted to apply: a first energy dose to the mixture to de-encapsulate the second component from capsules of the first subset, and/or a second energy dose to the mixture to de-encapsulate the second component from capsules of the second subset, the first and second energy doses being distinct.

For some implementations, the system further includes an imaging system, adapted to image the mixture at the anatomical site.

For some implementations, the imaging system includes an ultrasound transducer adapted to apply low-frequency ultrasound.

For some implementations, the de-encapsulation device and the imaging system include a single ultrasound transducer, adapted to apply high-frequency ultrasound to the mixture for de-encapsulation of the second component, and to apply low frequency ultrasound to the mixture for imaging thereof.

For some implementations, the system further includes a curing energy applicator, adapted to apply energy to the mixture for curing thereof subsequently to interaction of the second component with the first component.

For some implementations, the curing energy applicator includes an ultraviolet light source adapted to apply to the mixture electromagnetic energy in the ultraviolet region.

For some implementations, the curing energy applicator includes an infrared light source adapted to apply to the mixture electromagnetic energy in the infrared region.

For some implementations, the curing energy applicator includes a heating element adapted to heat the mixture.

For some implementations, the curing energy applicator includes an electrode adapted to apply electrical energy to the mixture.

There is further provided, in accordance with some implementations, a system for use at an anatomical site of a real or simulated subject, the system including: a mixture including a first component and a second component, the second component being encapsulated in frangible capsules that: prevent interaction of the second component with the first component, and/or are rupturable to expose the second component to the first component responsively to application of energy to the frangible capsules; and/or a delivery tool adapted to deliver the mixture toward the anatomical site.

For some implementations, at least one of the mixture, and the delivery tool is sterile.

For some implementations, the frangible capsules include frangible polymer capsules.

For some implementations, the frangible capsules include frangible protein capsules.

For some implementations, the frangible capsules include frangible lipid bi-layer capsules.

For some implementations, the first component is a fluid medium, having suspended therein the frangible capsules.

For some implementations, the mixture further includes a suspension medium in which the first component and the frangible capsules are suspended.

For some implementations, the mixture includes a powder including the first component and the frangible capsules.

For some implementations, the first component and the second component are two components of a two-part foam.

For some implementations, the first component and the second component are two components of a two-part polyurethane adhesive.

For some implementations, the first component and the second component are two components of a two-part resin.

For some implementations, the first component and the second component are two components of a two-part epoxy resin.

For some implementations, the system further includes a de-encapsulation device adapted to rupture the frangible capsules by applying energy to the frangible capsules.

For some implementations: the frangible capsules are second frangible capsules, the first component is encapsulated in first frangible capsules, and/or the de-encapsulation device is adapted to expose the second component to the first component by applying energy to the first frangible capsules and to the second frangible capsules.

For some implementations, the mixture further includes a suspension medium in which both the first frangible capsules and the second frangible capsules are suspended.

For some implementations, the mixture includes a powder including the first frangible capsules and the second frangible capsules.

For some implementations, the mixture further includes a third component.

For some implementations, the third component includes a reinforcing component adapted for mechanically reinforcing a repair structure formed by the first component and the second component, subsequent to exposure of the second component by the de-encapsulation device.

For some implementations, the third component includes an indicator component adapted to enable visualizing of a location of the mixture within the subject, during or following delivery of the mixture by the delivery tool.

For some implementations, the third component includes a surfactant.

For some implementations, the third component includes a medicament.

For some implementations, the medicament is an anti-inflammatory medicament.

For some implementations, the medicament has tissue-growth-promoting properties.

For some implementations, the medicament has antimicrobial properties.

For some implementations, the delivery tool includes a catheter, adapted to be transluminally advanced, with the mixture, to the anatomical site.

For some implementations, the catheter defines a lumen, the catheter being adapted to have the mixture advanced to the anatomical site via the lumen.

For some implementations, the system further includes a delivery structure containing the mixture, the delivery structure adapted to be transluminally advanced to the anatomical site via the catheter, with the mixture disposed therein.

For some implementations, the delivery structure includes a cavity accommodating the mixture.

For some implementations, the delivery structure includes a balloon.

For some implementations, the delivery structure includes a lattice structure holding the mixture.

For some implementations, the lattice structure is configured to be transluminally advanced via the catheter, and to automatically self-expand upon release from the catheter.

For some implementations, the lattice structure is configured to remain at the anatomical site, subsequent to exposure of the second component by the de-encapsulation device.

For some implementations: the system further includes a grasper, configured to be advanced transluminally via the delivery tool, and to hold the lattice structure in contact with tissue of the anatomical site; and/or the de-encapsulation device is configured to expose the second component to the first component by de-encapsulating the second component while the grasper holds the lattice structure in contact with tissue of the anatomical site.

For some implementations, the grasper is configured to be withdrawn from the subject via the delivery tool, subsequent to exposure of the second component by the de-encapsulation device.

For some implementations: the system is configured to de-encapsulate the second component while the grasper holds the lattice structure in contact with tissue of the anatomical site, such that the lattice structure adheres to tissue of the anatomical site.

For some implementations: the anatomical site is at a real or simulated native valve of a real or simulated heart of the subject; the grasper is configured to hold the lattice structure in contact with real or simulated leaflets of the real or simulated native valve; and/or the lattice structure is configured to remain adhered to the real or simulated leaflets of the real or simulated native valve, subsequent to exposure of the second component by the de-encapsulation device.

For some implementations, the delivery structure includes an implant adapted to be implanted at the anatomical site such that the delivery structure remains within the subject subsequently to exposure of the second component to the first component.

For some implementations, the delivery structure is absorbed into the subject subsequently to exposure of the second component to the first component.

For some implementations, the delivery structure is adapted to be removed from the subject subsequently to exposure of the second component to the first component.

For some implementations, the delivery tool includes a tool for applying the mixture to skin of the subject.

For some implementations, the delivery tool includes a tool for surgically delivering the mixture to the anatomical site.

For some implementations, the delivery tool includes an injection tool for injecting the mixture into the subject.

For some implementations, the delivery tool includes a subcutaneous delivery tool.

For some implementations, the de-encapsulation device includes a chemical adapted, upon introduction into the mixture, to carry out a chemical reaction resulting in de-encapsulating of the second component.

For some implementations, the de-encapsulation device includes an energy applicator adapted to apply energy to the mixture.

For some implementations, the energy applicator includes an electrode adapted to apply electrical energy to the mixture.

For some implementations, the energy applicator includes an electromagnetic radiation source adapted to apply electromagnetic energy to the mixture.

For some implementations, the energy applicator includes an ultrasound transducer.

For some implementations, the ultrasound transducer is adapted to apply high frequency ultrasound energy.

For some implementations, the ultrasound transducer is adapted to apply high-intensity focused ultrasound (HIFU) energy.

For some implementations, the ultrasound transducer is an intracorporeal ultrasound transducer.

For some implementations, the ultrasound transducer is an extracorporeal ultrasound transducer.

For some implementations, the first component of the mixture is also encapsulated, and the ultrasound transducer is adapted to apply ultrasound energy at a single frequency to de-encapsulate the first component and the second component.

For some implementations, the first component is also encapsulated, and the ultrasound transducer is adapted to apply ultrasound energy at a first frequency to de-encapsulate the first component and at a second frequency to de-encapsulate the second component.

For some implementations, a first subset of the frangible capsules includes frangible capsules having a first degree of strength, and a second subset of the frangible capsules includes frangible capsules having a second degree of strength, and/or the de-encapsulation device is adapted to apply: a first energy dose to the mixture to de-encapsulate the second component from frangible capsules of the first subset, and/or a second energy dose to the mixture to de-encapsulate the second component from frangible capsules of the second subset, the first and second energy doses being distinct.

For some implementations, the system further includes an imaging system, adapted to image the mixture at the anatomical site.

For some implementations, the imaging system includes an ultrasound transducer adapted to apply low-frequency ultrasound.

For some implementations, the de-encapsulation device and the imaging system include a single ultrasound transducer, adapted to apply high-frequency ultrasound to the mixture for de-encapsulation of the second component, and to apply low frequency ultrasound to the mixture for imaging thereof.

For some implementations, the system further includes a curing energy applicator, adapted to apply energy to the mixture for curing thereof subsequently to exposure of the second component to the first component.

For some implementations, the curing energy applicator includes an ultraviolet light source adapted to apply to the mixture electromagnetic energy in the ultraviolet region.

For some implementations, the curing energy applicator includes an infrared light source adapted to apply to the mixture electromagnetic energy in the infrared region.

For some implementations, the curing energy applicator includes a heating element adapted to heat the mixture.

For some implementations, the curing energy applicator includes an electrode adapted to apply electrical energy to the mixture.

There is further provided, in accordance with some implementations, a method, including: delivering, to an anatomical site of a real or simulated subject, a mixture including: a first component, and/or a second component, encapsulated in a manner that prevents its interaction with the first component; and/or while the mixture remains at the anatomical site, exposing the second component to the first component by de-encapsulating the second component.

For some implementations, the method further includes sterilizing the mixture.

For some implementations, the first component is a fluid medium, having suspended therein the encapsulated second component.

For some implementations, the mixture further includes a suspension medium in which the first component and the encapsulated second component are suspended, and delivering the mixture includes delivering the mixture that further includes the suspension medium.

For some implementations, the mixture includes a powder including the first component and the encapsulated second component, and delivering the mixture includes delivering the powder.

For some implementations: the first component is encapsulated in first capsules, the encapsulation of the second component being encapsulation in second capsules, and/or exposing the second component to the first component includes de-encapsulating both the first component and the second component.

For some implementations, the mixture further includes a third component, and delivering the mixture includes delivering the mixture that further includes the third component.

For some implementations, the third component includes a reinforcing component, the method further including using the reinforcing component, mechanically reinforcing a repair structure formed by the first component and the second component, subsequent to exposing of the second component.

For some implementations, the third component includes an indicator component, the method further including visualizing a location of the mixture within the subject, during or following delivering of the mixture, by visualizing of the indicator component.

For some implementations, the third component includes a surfactant, and delivering the mixture includes delivering the mixture that further includes the surfactant.

For some implementations, the third component includes a medicament, and delivering the mixture includes delivering the mixture that further includes the medicament.

For some implementations, the medicament is an anti-inflammatory medicament, and delivering the mixture includes delivering the mixture that further includes the anti-inflammatory medicament.

For some implementations, the medicament is a tissue-growth-promoting medicament, and delivering the mixture includes delivering the mixture that further includes the tissue-growth-promoting medicament.

For some implementations, the medicament is an antimicrobial medicament, and delivering the mixture includes delivering the mixture that further includes the antimicrobial medicament.

For some implementations, delivering includes applying the mixture to skin of the subject.

For some implementations, delivering includes surgically delivering the mixture to the anatomical site.

For some implementations, delivering includes subcutaneously delivering the mixture to the anatomical site.

For some implementations, delivering includes transluminally advancing the mixture to the anatomical site.

For some implementations, transluminally advancing includes transluminally advancing the mixture to the anatomical site within a delivery structure containing the mixture.

For some implementations, the method further includes removing the delivery structure from the subject subsequently to exposing the second component to the first component.

For some implementations, advancing the delivery structure to the anatomical site includes implanting the delivery structure at the anatomical site such that the delivery structure remains within the subject subsequently to exposing the second component to the first component.

For some implementations: transluminally advancing includes transluminally advancing the mixture to the anatomical site via a catheter; and/or the method further includes, subsequently to exposing the second component to the first component, removing the catheter from the subject such that the delivery structure remains within the subject.

For some implementations, delivering includes injecting the mixture into the subject.

For some implementations, injecting includes injecting the mixture to the anatomical site.

For some implementations, injecting includes injecting the mixture into a lumen of the subject having a fluid flow therethrough, at a location remote from the anatomical site, the mixture flowing with the fluid via the lumen to the anatomical site.

For some implementations, delivering the mixture to the anatomical site includes delivering the mixture to a real or simulated heart of the subject.

For some implementations, delivering the mixture to the anatomical site includes delivering the mixture to a real or simulated valve of the heart of the subject.

For some implementations, de-encapsulating includes introducing into the mixture a chemical which carries out a chemical reaction resulting in de-encapsulating of the second component.

For some implementations, de-encapsulating includes applying energy to the mixture.

For some implementations, applying the energy includes applying electrical energy to the mixture.

For some implementations, applying the energy includes applying electromagnetic energy to the mixture.

For some implementations, applying the energy includes applying ultrasound energy to the mixture.

For some implementations, applying ultrasound energy includes applying high-frequency ultrasound energy.

For some implementations, applying ultrasound energy includes applying high-intensity focused ultrasound (HIFU) energy.

For some implementations, applying ultrasound energy includes applying ultrasound energy from an ultrasound transducer disposed within the subject.

For some implementations, applying ultrasound energy includes applying ultrasound energy from an ultrasound transducer exterior to the subject.

For some implementations, the first component is also encapsulated, and applying ultrasound energy includes applying ultrasound energy at a single frequency to de-encapsulate the first component and the second component.

For some implementations, the first component is also encapsulated, and applying ultrasound energy includes applying ultrasound energy at a first frequency to de-encapsulate the first component and at a second frequency to de-encapsulate the second component.

For some implementations, the method further includes mixing the first component and the encapsulated second component to form the mixture.

For some implementations, the method further includes encapsulating the second component within capsules.

For some implementations, encapsulating includes encapsulating the second component within polymer capsules.

For some implementations, encapsulating includes encapsulating the second component within protein capsules.

For some implementations, encapsulating includes encapsulating the second component within lipid bilayer capsules.

For some implementations, encapsulating includes encapsulating a first portion of the second component in capsules having a first degree of strength, and encapsulating a second portion of the second component in capsules having a second degree of strength, wherein de-encapsulating includes applying energy to the mixture to de-encapsulate the second component, and the first and second degrees of strength require distinct energy doses for de-encapsulation.

For some implementations, the method further includes, prior to de-encapsulating, imaging the mixture at the anatomical site.

For some implementations, imaging the mixture at the anatomical site includes applying imaging ultrasound energy to the mixture at the anatomical site.

For some implementations, de-encapsulating includes applying de-encapsulating ultrasound energy to the mixture, the de-encapsulating ultrasound energy having a frequency that is higher than that of the imaging ultrasound energy.

For some implementations, applying the imaging ultrasound energy and applying the de-encapsulating ultrasound energy include applying the imaging ultrasound energy and applying the de-encapsulating ultrasound energy using a single ultrasound transducer.

For some implementations, applying the de-encapsulating ultrasound energy includes applying the de-encapsulating ultrasound energy responsively to imaging the mixture at the anatomical site.

For some implementations, the method further includes, following exposing the second component to the first component, curing the mixture by applying curing-energy to the mixture.

For some implementations, applying curing-energy includes illuminating the mixture with ultraviolet light.

For some implementations, applying curing-energy includes illuminating the mixture with infrared light.

For some implementations, applying curing-energy includes heating the mixture.

For some implementations, applying curing-energy includes applying electrical energy to the mixture.

For some implementations, the first component and the second component are two components of a two-part resin, and delivering the mixture includes delivering the two components of the two-part resin.

For some implementations, the first component and the second component are two components of a two-part foam, and delivering the mixture includes delivering the two components of the two-part foam.

For some implementations, the first component and the second component are two components of a two-part polyurethane adhesive, and delivering the mixture includes delivering the two components of the two-part polyurethane adhesive.

For some implementations: exposing the second component to the first component forms a repair structure, and/or the method further including using the repair structure, anchoring an implant at the anatomical site.

For some implementations: exposing the second component to the first component forms a repair structure, and/or the method further including using the repair structure, augmenting anchoring of an implant at the anatomical site.

For some implementations: exposing the second component to the first component forms a repair structure, and/or the method further including using the repair structure, reshaping tissue at the anatomical site.

For some implementations: exposing the second component to the first component forms a repair structure, and/or the method further including using the repair structure, adhering tissue at the anatomical site.

For some implementations, exposing the second component to the first component forms a repair structure, and/or the method further including using the repair structure, adhering an implant to the anatomical site.

For some implementations, exposing the second component to the first component includes forming a repair structure within an implant at the anatomical site, the repair structure inflating the implant at the anatomical site.

For some implementations, forming the repair structure includes inflating a coaptation device improving coaptation of leaflets of a real or simulated heart valve of the subject.

For some implementations, forming the repair structure includes inflating a spacer improving function of a real or simulated heart valve of the subject.

There is further provided, in accordance with some implementations, a system for use at an anatomical site of a real or simulated subject, the system including: a mixture including a first component and a second component, the second component being encapsulated in capsules in a manner that prevents its interaction with the first component; a delivery tool adapted to deliver the mixture toward the anatomical site; and/or a de-encapsulation device adapted to expose the second component to the first component by de-encapsulating the second component.

For some implementations, at least one of the mixture, the delivery tool and the de-encapsulation device is sterile.

For some implementations, the system further includes a mixing device adapted

to mix the first component and the encapsulated second component to form the mixture.

For some implementations, the capsules include polymer capsules.

For some implementations, the capsules include protein capsules.

For some implementations, the capsules include lipid bi-layer capsules.

For some implementations, the first component is a fluid medium, having suspended therein the capsules.

For some implementations, the mixture further includes a suspension medium in which the first component and the capsules are suspended.

For some implementations, the mixture includes a powder including the first component and the capsules.

For some implementations, the first component and the second component are two components of a two-part foam.

For some implementations, the first component and the second component are two components of a two-part polyurethane adhesive.

For some implementations, the first component and the second component are two components of a two-part resin.

For some implementations, the first component and the second component are two components of a two-part epoxy resin.

For some implementations, the capsules are second capsules, and the first component is encapsulated in first capsules.

For some implementations, the de-encapsulation device is adapted to expose the first component and the second component to each other, by de-encapsulating the first component and the second component.

For some implementations, the de-encapsulation device includes: a first de-encapsulation device adapted to expose the first component by de-encapsulating the first component from the first capsules; and/or a second de-encapsulation device adapted to expose the second component by de-encapsulating the second component from the second capsules, such that the first component and the second component are exposed to each other.

For some implementations, the mixture further includes a suspension medium in which both the first capsules and the second capsules are suspended.

For some implementations, the mixture includes a powder including the first capsules and the second capsules.

For some implementations, the mixture further includes a third component.

For some implementations, the third component includes a reinforcing component adapted for mechanically reinforcing a repair structure formed by the first component and the second component, subsequent to exposure of the second component by the de-encapsulation device.

For some implementations, the third component includes an indicator component adapted to enable visualizing of a location of the mixture within the subject, during or following delivery of the mixture by the delivery tool.

For some implementations, the third component includes a surfactant.

For some implementations, the third component includes a medicament.

For some implementations, the medicament is an anti-inflammatory medicament.

For some implementations, the medicament has tissue-growth-promoting properties.

For some implementations, the medicament has antimicrobial properties.

For some implementations, the delivery tool includes a catheter, adapted to be transluminally advanced, with the mixture, to the anatomical site.

For some implementations, the catheter defines a lumen, the catheter being adapted to have the mixture advanced to the anatomical site via the lumen.

For some implementations, the system further includes a delivery structure containing the mixture, the delivery structure adapted to be transluminally advanced to the anatomical site via the catheter, with the mixture disposed therein.

For some implementations, the delivery structure includes a lattice structure holding the mixture.

For some implementations, the lattice structure is configured to be transluminally advanced via the catheter, and to automatically self-expand upon release from the catheter.

For some implementations, the lattice structure is configured to remain at the anatomical site, subsequent to exposure of the second component by the de-encapsulation device.

For some implementations: the system further includes a grasper, configured to be advanced transluminally via the delivery tool, and to hold the lattice structure in contact with tissue of the anatomical site; and/or the de-encapsulation device is configured to expose the second component to the first component by de-encapsulating the second component while the grasper holds the lattice structure in contact with tissue of the anatomical site.

For some implementations, the grasper is configured to be withdrawn from the subject via the delivery tool, subsequent to exposure of the second component by the de-encapsulation device.

For some implementations: the system is configured to de-encapsulate the second

component while the grasper holds the lattice structure in contact with tissue of the anatomical site such that the lattice structure adheres to tissue of the anatomical site.

For some implementations: the anatomical site is at a native valve of a real or simulated heart of the subject; the grasper is configured to hold the lattice structure in contact with leaflets of the native valve; and/or the lattice structure is configured to remain adhered to leaflets of the native valve, subsequent to exposure of the second component by the de-encapsulation device.

For some implementations, the delivery structure includes a cavity accommodating the mixture.

For some implementations, the delivery structure includes a balloon.

For some implementations, the delivery structure includes an implant adapted to be implanted at the anatomical site such that the delivery structure remains within the subject subsequently to exposure of the second component to the first component.

For some implementations, the delivery structure is absorbed into the subject subsequently to exposure of the second component to the first component.

For some implementations, the delivery structure is adapted to be removed from the subject subsequently to exposure of the second component to the first component.

For some implementations, the delivery tool includes a tool for applying the mixture to skin of the subject.

For some implementations, the delivery tool includes a tool for surgically delivering the mixture to the anatomical site.

For some implementations, the delivery tool includes an injection tool for injecting the mixture into the subject.

For some implementations, the delivery tool includes a subcutaneous delivery tool.

For some implementations, the de-encapsulation device includes a chemical adapted, upon introduction into the mixture, to carry out a chemical reaction resulting in de-encapsulating of the second component.

For some implementations, the de-encapsulation device includes an energy applicator adapted to apply energy to the mixture.

For some implementations, the energy applicator includes an electrode adapted to apply electrical energy to the mixture.

For some implementations, the energy applicator includes an electromagnetic radiation source adapted to apply electromagnetic energy to the mixture.

For some implementations, the energy applicator includes an ultrasound transducer.

For some implementations, the ultrasound transducer is adapted to apply high frequency ultrasound energy.

For some implementations, the ultrasound transducer is adapted to apply high-intensity focused ultrasound (HIFU) energy.

For some implementations, the ultrasound transducer is an intracorporeal ultrasound transducer.

For some implementations, the ultrasound transducer is an extracorporeal ultrasound transducer.

For some implementations, the first component of the mixture is also encapsulated, and the ultrasound transducer is adapted to apply ultrasound energy at a single frequency to de-encapsulate the first component and the second component.

For some implementations, the first component is also encapsulated, and the ultrasound transducer is adapted to apply ultrasound energy at a first frequency to de-encapsulate the first component and at a second frequency to de-encapsulate the second component.

For some implementations, a first subset of the capsules includes capsules having a first degree of strength, and a second subset of the capsules includes capsules having a second degree of strength, and/or the de-encapsulation device is adapted to apply: a first energy dose to the mixture to de-encapsulate the second component from capsules of the first subset, and/or a second energy dose to the mixture to de-encapsulate the second component from capsules of the second subset, the first and second energy doses being distinct.

For some implementations, the system further includes an imaging system, adapted to image the mixture at the anatomical site.

For some implementations, the imaging system includes an ultrasound transducer adapted to apply low-frequency ultrasound.

For some implementations, the de-encapsulation device and the imaging system include a single ultrasound transducer, adapted to apply high-frequency ultrasound to the mixture for de-encapsulation of the second component, and to apply low frequency ultrasound to the mixture for imaging thereof.

For some implementations, the system further includes a curing energy applicator, adapted to apply energy to the mixture for curing thereof subsequently to exposure of the second component to the first component.

For some implementations, the curing energy applicator includes an ultraviolet light source adapted to apply to the mixture electromagnetic energy in the ultraviolet region.

For some implementations, the curing energy applicator includes an infrared light source adapted to apply to the mixture electromagnetic energy in the infrared region.

For some implementations, the curing energy applicator includes a heating element adapted to heat the mixture.

For some implementations, the curing energy applicator includes an electrode adapted to apply electrical energy to the mixture.

There is further provided, in accordance with some implementations, a system for treating a real or simulated subject, the system including a composition including a mixture that includes: a first component, and/or a second component, encapsulated in a manner that prevents its interaction with the first component.

For some implementations, the mixture is sterile.

For some implementations, the composition is an adhesive.

For some implementations, the composition is configured such that interaction of the second component with the first component forms an adhesive.

For some implementations, the first component is a fluid medium, having suspended therein the encapsulated second component.

For some implementations, the system further includes a suspension medium in which the first component and the encapsulated second component are suspended.

For some implementations, the composition is configured such that interaction of the second component with the first component hardens the composition.

For some implementations: the system further includes an implant for implantation in the subject, and/or wherein the composition is held by the implant.

For some implementations, the composition is held inside the implant.

For some implementations, the composition is held on the implant.

For some implementations, the system further includes a medical repair

structure, formed by de-encapsulation of the second component to facilitate interaction between the first component and the second component.

For some implementations, the medical repair structure includes an implant.

For some implementations, the medical repair structure is adapted for anchoring an implant at an anatomical site of a real or simulated subject.

For some implementations, the medical repair structure is adapted for augmenting anchoring of an implant at an anatomical site of a real or simulated subject.

For some implementations, the medical repair structure is adapted for reshaping tissue at an anatomical site.

For some implementations, the medical repair structure is adapted for adhesion of, or to, tissue at an anatomical site.

For some implementations, the medical repair structure is adapted for adhesion of an implant at an anatomical site.

For some implementations, the medical repair structure is adapted to inflate an implant at an anatomical site.

For some implementations, the medical repair structure forms a coaptation device adapted to improve coaptation of leaflets of a real or simulated heart valve of a real or simulated subject.

For some implementations, the medical repair structure includes a spacer adapted to improve function of a real or simulated heart valve of the subject.

For some implementations, the composition is a liquid.

For some implementations, the composition is viscoplastic.

For some implementations, the composition is viscoelastic.

For some implementations, the composition is a powder.

For some implementations, the composition is a paste.

For some implementations, the composition is a gel.

For some implementations, the composition is a hydrogel.

For some implementations, the first component is encapsulated in first capsules, and the second component is encapsulated in second capsules.

For some implementations, the composition further includes a suspension

medium in which both the first capsules and the second capsules are suspended.

For some implementations, the composition further includes a powder including the first capsules and the second capsules.

For some implementations, the first capsules are microcapsules.

For some implementations, the second capsules are microcapsules.

For some implementations, the composition further includes a third component.

For some implementations, the third component includes a reinforcing component adapted to mechanically reinforce the composition, when it is hardened.

For some implementations, the third component includes an indicator component adapted to enable visualizing of the composition within the subject.

For some implementations, the third component includes a surfactant.

For some implementations, the third component includes a medicament.

For some implementations, the medicament is an anti-inflammatory medicament.

For some implementations, the medicament is an antimicrobial medicament.

For some implementations, the second component is adapted to be de-encapsulated by application of energy to the composition.

For some implementations, the second component is adapted to be de-encapsulated by application of high-frequency ultrasound energy to the mixture.

For some implementations, the second component is adapted to be de-encapsulated by application of high-intensity focused ultrasound (HIFU) energy to the mixture.

For some implementations, the second component is adapted to be de-encapsulated by application of electrical energy to the mixture.

For some implementations, the second component is adapted to be de-encapsulated by application of electromagnetic energy to the mixture.

For some implementations, the second component is adapted to be de-encapsulated by introduction of a chemical agent into the mixture, to carry out a chemical reaction resulting in de-encapsulating of the second component.

For some implementations, the second capsules include polymer capsules.

For some implementations, the second capsules encapsulating the second component include protein capsules.

For some implementations, the second capsules encapsulating the second component include lipid bi-layer capsules.

For some implementations: the second component is configured to interact with the first component upon the second component being de-encapsulated, and/or the composition is curable, subsequently to de-encapsulating of the second component, to form a repair structure.

For some implementations, the composition is curable by application of energy thereto.

For some implementations, the composition is curable by application of ultraviolet light thereto.

For some implementations, the composition is curable by application of infrared light thereto.

For some implementations, the composition is curable by application of heat thereto.

For some implementations, the first component and the second component are two components of a two-part foam.

For some implementations, the first component and the second component are two components of a two-part polyurethane adhesive.

For some implementations, the first component and the second component are two components of a two-part resin.

For some implementations, the first component and the second component are two components of a two-part epoxy resin.

There is further provided, in accordance with some implementations, a system, including: a medical implant, configured to be implanted in a real or simulated subject; and/or a composition, held by the medical implant, the composition including a mixture that includes: a first component, and/or a second component, encapsulated in capsules in a manner that prevents its interaction with the first component.

For some implementations, at least one of the medical implant and the composition is sterile.

For some implementations, the composition is held inside the implant.

For some implementations, the composition is held on the implant.

For some implementations, the composition is an adhesive.

For some implementations, the composition is configured such that interaction of the second component with the first component forms an adhesive.

For some implementations, the composition is configured such that interaction of the second component with the first component hardens the composition.

For some implementations, the composition is a liquid.

For some implementations, the composition is viscoplastic.

For some implementations, the composition is viscoelastic.

For some implementations, the composition is a powder.

For some implementations, the composition is a paste.

For some implementations, the composition is a gel.

For some implementations, the composition is a hydrogel.

For some implementations, the first component is a fluid medium, having suspended therein the encapsulated second component.

For some implementations, the composition further includes a suspension medium in which the first component and the capsules encapsulating the second component are suspended.

For some implementations, the first component is encapsulated in first capsules, and the capsules encapsulating the second component are second capsules.

For some implementations, the composition further includes a suspension medium in which both the first capsules and the second capsules are suspended.

For some implementations, the composition further includes a powder including the first capsules and the second capsules.

For some implementations, the first capsules and/or the second capsules are microcapsules.

For some implementations, the composition further includes a third component.

For some implementations, the third component includes a reinforcing component adapted to mechanically reinforce the composition, when it is hardened.

For some implementations, the third component includes an indicator component adapted to enable visualizing of the composition within the subject.

For some implementations, the third component includes a surfactant.

For some implementations, the third component includes a medicament.

For some implementations, the medicament is an anti-inflammatory medicament.

For some implementations, the medicament is an antimicrobial medicament.

For some implementations, the second component is adapted to be de-encapsulated by application of energy to the mixture.

For some implementations, the second component is adapted to be de-encapsulated by application of high-frequency ultrasound energy to the mixture.

For some implementations, the second component is adapted to be de-encapsulated by application of high-intensity focused ultrasound (HIFU) energy to the mixture.

For some implementations, the second component is adapted to be de-encapsulated by application of electrical energy to the mixture.

For some implementations, the second component is adapted to be de-encapsulated by application of electromagnetic energy to the mixture.

For some implementations, the second component is adapted to be de-encapsulated by introduction of a chemical agent into the mixture, to carry out a chemical reaction resulting in de-encapsulating of the second component.

For some implementations, the capsules encapsulating the second component include polymer capsules.

For some implementations, the capsules encapsulating the second component include protein capsules.

For some implementations, the capsules encapsulating the second component include lipid bi-layer capsules.

For some implementations, the composition is curable, subsequently to de-encapsulating of the second component, to form a repair structure.

For some implementations, the composition is curable by application of energy thereto.

For some implementations, the composition is curable by application of ultraviolet light thereto.

For some implementations, the composition is curable by application of infrared light thereto.

For some implementations, the composition is curable by application of heat thereto.

For some implementations, the first component and the second component are two components of a two-part foam.

For some implementations, the first component and the second component are two components of a two-part polyurethane adhesive.

For some implementations, the first component and the second component are two components of a two-part resin.

For some implementations, the first component and the second component are two components of a two-part epoxy resin.

For some implementations, the medical implant includes a lattice structure holding the mixture.

For some implementations, the lattice structure is configured to remain implanted in the subject, subsequently to interaction of the second component with the first component.

For some implementations, the system further includes: a grasper, configured to be advanced transluminally to an anatomical site of the subject, and to hold the lattice structure in contact with tissue of the subject at the anatomical site; and/or a de-encapsulation device configured to expose the second component to the first component by de-encapsulating the second component while the grasper holds the lattice structure in contact with the tissue at the anatomical site.

For some implementations, the grasper is configured to be transluminally withdrawn from the subject, subsequently to exposure of the second component by the de-encapsulation device.

For some implementations, the system is configured such that de-encapsulating the second component while the grasper holds the lattice structure in contact with tissue of the anatomical site adheres the lattice structure to tissue at the anatomical site.

For some implementations: the anatomical site is at a native valve of a real or simulated heart of the subject; the grasper is configured to hold the lattice structure in contact with leaflets of the native valve; and/or the lattice structure is configured to remain adhered to leaflets of the native valve, subsequent to exposure of the second component by the de-encapsulation device.

There is further provided, in accordance with some implementations, a system at an anatomical site of a real or simulated subject, the system including: an implant, including: a body, and/or a composition, held by the body, the composition including a mixture that includes: a first component, and/or a second component, encapsulated in capsules in a manner that prevents its interaction with the first component; and/or a delivery tool, configured to deliver the implant to the anatomical site, and including a grasper that is configured to hold the body in contact with tissue at the anatomical site.

For some implementations, at least one of the implant and the delivery tool is sterile.

There is further provided, in accordance with some implementations, a system at an anatomical site of a real or simulated subject, the system including an implant, including: a porous matrix, and/or a composition, disposed on the matrix, the composition including a mixture that includes: a first component, and/or a second component, encapsulated in capsules in a manner that prevents its interaction with the first component.

For some implementations, at least one of the porous matrix and the composition is sterile.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

The present invention will be more fully understood from the following detailed description of applications thereof, taken together with the drawings, in which:

### BRIEF DESCRIPTION OF THE DRAWINGS

FIGS. 1A-1F are schematic illustrations of multi-component compositions, in accordance with some implementations;

FIGS. 2A-C are schematic illustrations of capsules forming part of the multi-component compositions, according to some implementations;

FIG. 3 is a schematic illustration of a method for delivering the multi-component compositions to an anatomical site of a subject, according to some implementations;

FIGS. 4A-E are schematic illustrations of mechanisms for delivering the multi-component compositions to the anatomical site of the subject, according to some implementations;

FIGS. 5A-D are schematic illustrations of steps of a method of forming an implant using the multi-component compositions, according to some implementations;

FIGS. 6A-D are schematic illustrations of steps of a method of forming and anchoring an implant using the multi-component compositions, according to some implementations;

FIGS. 7A-C are schematic illustrations of a system including a tissue anchor, in accordance with some implementations;

FIGS. 8A-C are schematic illustrations of steps of implanting and anchoring a tissue anchor using a multi-component compositions, according to some implementations; and

FIGS. 9A-I are schematic illustrations of steps of adhering leaflets of a heart valve to each other using the multi-component compositions, according to some implementations.

### DETAILED DESCRIPTION

The principles of the tissue anchors may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the invention is not limited in its application to the details of construction and the arrangement of the components set forth in the following description or illustrated in the drawings. The invention is capable of other implementations or of being practiced or carried out in various ways. Furthermore, it is to be understood that the phraseology and terminology employed in the disclosure is for the purpose of description and should not be regarded as limiting.

For the purposes of this application, the term “subject” relates to any mammal, particularly humans.

For the purposes of this application, the terms “capsule” and “microcapsule” are used interchangeably, and relate to capsules having a longest dimension in the range of 10 nm to 10 μm. In some implementations, microcapsules may have a longest dimension in the range of in the range of 10 nm to 1 μm, in the range of 10 nm to 500 nm, in the range of 10 nm to 100 nm, or in the range of 1 μm to 10 μm, in the range of 1 μm to 5 μm, in the range of 500 nm to 5 μm, in the range of 500 nm to 2.5 μm, in the range of 500 nm to 1 μm, in the range of 100 nm to 1 μm, or in the range of 100 nm to 500 nm.

For the purposes of this application, the terms “encapsulated” and “microencapsulated” are used interchangeably, and relate to encapsulation in capsules having a longest dimension in the range of 10 nm to 1 mm.

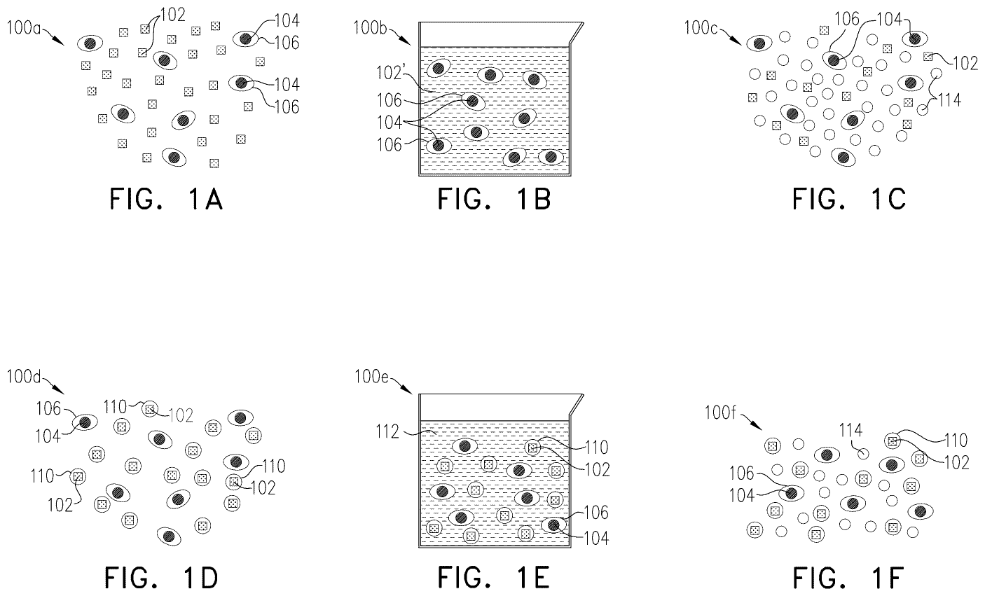

Reference is now made to FIGS. 1A, 1B, 1C, 1D, 1E, and 1F, which are schematic illustrations of multi-component compositions 100a, 100b, 100c, 100d, 100e, 100f, each of which can be considered variants of a composition 100 for treating a subject, in accordance with some implementations of the invention.

As seen, FIGS. 1A to 1F schematically illustrate respective compositions 100a, 100b, 100c, 100d, 100e, 100f, each of which comprises a mixture. The mixture includes a first component 102, 102′, and a second component 104 that is encapsulated in frangible capsules 106. Encapsulation of second component 104 in capsules 106 prevents the interaction of the second component with first component 102.

In some implementations, first component 102 and second component 104 are two components of a two-part adhesive or a two-part resin, such as a two-part epoxy system (e.g., comprising an epoxy resin and a hardener or catalyst).

In some implementations, first component 102 and second component 104 are two components of a two-part foam.

In some implementations, first component 102 and second component 104 are two components of a two-part polyurethane adhesive.

In some implementations, and as illustrated in FIG. 1A, composition 100a is a powder, including first component 102 and capsules 106 encapsulating second component 104.

In some implementations, and as illustrated in FIG. 1B, a first component 102′ of composition 100b is a fluid (such as a liquid or fluid gel) or is a component of the fluid, such as being dissolved in the fluid, or is suspended in the fluid. Capsules 106 encapsulating second component 104 are suspended in first component 102′.

In some implementations, the composition further includes a third component 114. In some implementations, and as shown in FIG. 1C, composition 100c includes third component 114 in addition to unencapsulated first component 102 and encapsulated second component 104. In some implementations, and as shown in FIG. 1F, composition 100f includes third component 114, in addition to encapsulated first component 102 and an encapsulated second component 104.

In some implementations, third component 114 comprises a reinforcing component. The reinforcing component can mechanically reinforce a repair structure formed by the composition, in situ, for example when the composition is hardened, as explained in further detail hereinbelow.

In some implementations, third component 114 comprises an indicator component. The indicator component can enable visualizing of the composition within the body of a subject, as explained in further detail hereinbelow. For example, the indicator component can be, or include, a contrast material.

In some implementations, third component 114 comprises a foaming agent such as a surfactant or a blowing agent.

In some implementations, third component 114 comprises a medicament, such as an anti-inflammatory, a medicament having tissue-growth-promoting properties, or an antimicrobial (e.g., an antiseptic or an antibiotic) medicament.

Purely as an illustrative example, a three-part composition may comprise (i) an epoxy resin (which may or may not be encapsulated), (ii) a hardener (which may or may not be encapsulated), and (iii) an accelerator (which may or may not be encapsulated).

In some implementations, and as illustrated in FIGS. 1D to 1F, first component 102 may also be encapsulated, e.g., in frangible capsules 110. Capsules 110 are shown differently to capsules 106, to illustrate that capsules 110 may have different characteristics (e.g., chemical composition, physical characteristics, and/or dimensions) from capsules 106. However, in some implementations, capsules 110 are similar (e.g., identical) to capsules 106.

In some implementations, and as illustrated in FIG. 1D, composition 100d is a powder including first capsules 110 encapsulating first component 102, and capsules 106 encapsulating second component 104.

In some implementations, and as illustrated in FIG. 1E, composition 100e further includes a suspension medium 112 (e.g., a liquid, a gel, or a fluid gel), in which capsules 110 (encapsulating first component 102) and capsules 106 (encapsulating second components 104) are suspended.

As explained in further detail hereinbelow, at the appropriate time and location, second component 104 is de-encapsulated, for example by destruction of capsules 106, to enable interaction between first component 102 and the second component. In some implementations, in which first component 102 is encapsulated in first capsules 110, the first component must also be de-encapsulated to enable such interaction.

The following description can relate to any one or more of compositions 100a, 100b, 100c, 100d, 100e, and 100f.

In some implementations, the composition is an adhesive. In some implementations, the composition is configured such that interaction of second component 104 with first component 102 forms an adhesive.

In some implementations, the composition is configured such that interaction of second component 104 with first component 102 hardens the composition.

In some implementations, the composition is a liquid (e.g., is provided as a liquid). In some implementations, the composition is a fluid gel (e.g., is provided as a fluid gel). In some implementations, the composition is viscoelastic (e.g., is provided as viscoelastic). In some implementations, the composition is a powder (e.g., is provided as a powder). In some implementations, the composition is a paste (e.g., is provided as a paste). In some implementations, the composition is a gel, such as a hydrogel (e.g., is provided as a gel, such as a hydrogel).

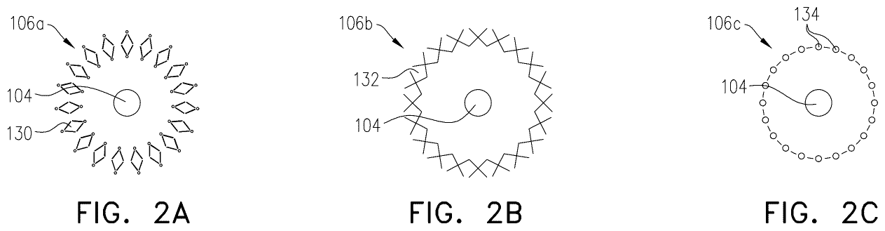

Reference is now made to FIGS. 2A, 2B, and 2C, which are schematic illustrations of capsules 106a, 106b, 106c forming part of the multi-component compositions of FIGS. 1A to 1F, according to some applications of the invention. FIGS. 2A to 2C are described herein as variants of capsules 106 encapsulating second component 104, but it is to be understood that the description may apply to any other capsules described herein, such as capsules 110, or capsules encapsulating third component 114.

In some implementations, and as illustrated in FIG. 2A, capsule 106a is formed of a bi-layer 130 of amphiphilic molecules, e.g., a lipid bilayer comprising phospholipids. As such, capsule 106a may be, or may be similar to, a liposome or a vesicle.

In some implementations, and as illustrated in FIG. 2B, capsule 106b encapsulating second component 104 is formed of a polymeric layer 132.

In some implementations, and as illustrated in FIG. 2C, capsule 106c encapsulating second component 104 is a protein capsule, formed of linked amino acids 134.

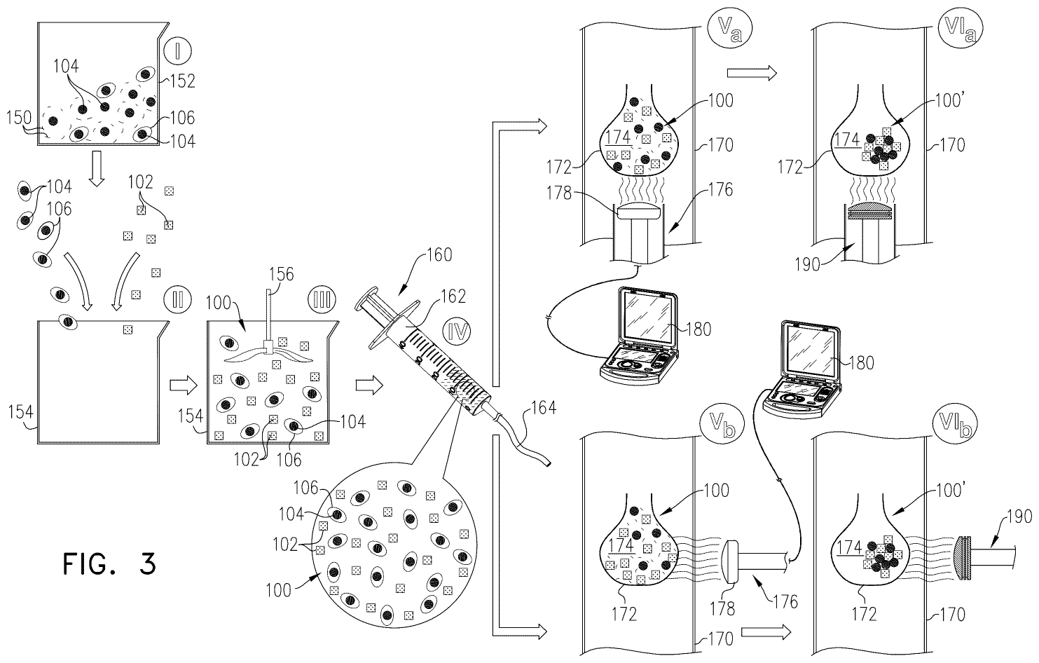

Reference is now made to FIG. 3, which is a schematic illustration of at least some steps of a method for delivering composition 100 to an anatomical site of a subject, according to some applications of the invention.

As seen in FIG. 3, at step I, second component 104 undergoes encapsulation into capsules 106. The encapsulation may be carried out using any mechanism known in the art. For example, suitable capsule components 150 can be mixed in a receptacle 152 with second component 104, such that capsules 106 form, e.g., around component 104. It is to be appreciated that, in applications in which the first component is also encapsulated (FIGS. 1D-F), step I can be carried out twice, once to form capsules 106 around second component 104, and again to form capsules 110 around first component 102.

At step II, first component 102 and second component 104 enclosed in capsules 106, are passed to a suitable receptacle 154, and are mixed at step III to form composition 100. In some implementations, such mixing may include adding a suspension fluid, or additional component such as third component 114 (FIGS. 1C and 1F) to the composition. In applications in which first component 102 is enclosed in first capsules 110 (see FIGS. 1D-F), the mixing step includes mixing the first capsules enclosing the first component with the second capsules enclosing the second component, as well as other components, as relevant. The mixing may be carried out using any suitable mechanism, manually or mechanically, e.g., by shaking, stirring (e.g., using a magnetic stir rod, a rotary cone mixing machine, a mixing syringe, etc.), or by any other suitable method.

It is to be appreciated that steps I to III are steps for manufacturing composition 100, including first component 102 and second component 104, where the second component is encapsulated in capsules 106. In some implementations, composition 100 may be provided pre-formed (e.g., pre-mixed). Thus, steps IV to VI may be performed independently from steps I to III (e.g., by a different individual and/or on a subsequent date).

At step IV, a delivery tool 160 is used to deliver composition 100 toward the anatomical site. In the example shown, delivery tool 160 is illustrated as a syringe 162 connected to a catheter 164, for delivery of the composition toward an anatomical site. For example, the composition may be transluminally and/or transcatheterally advanced to the anatomical site.

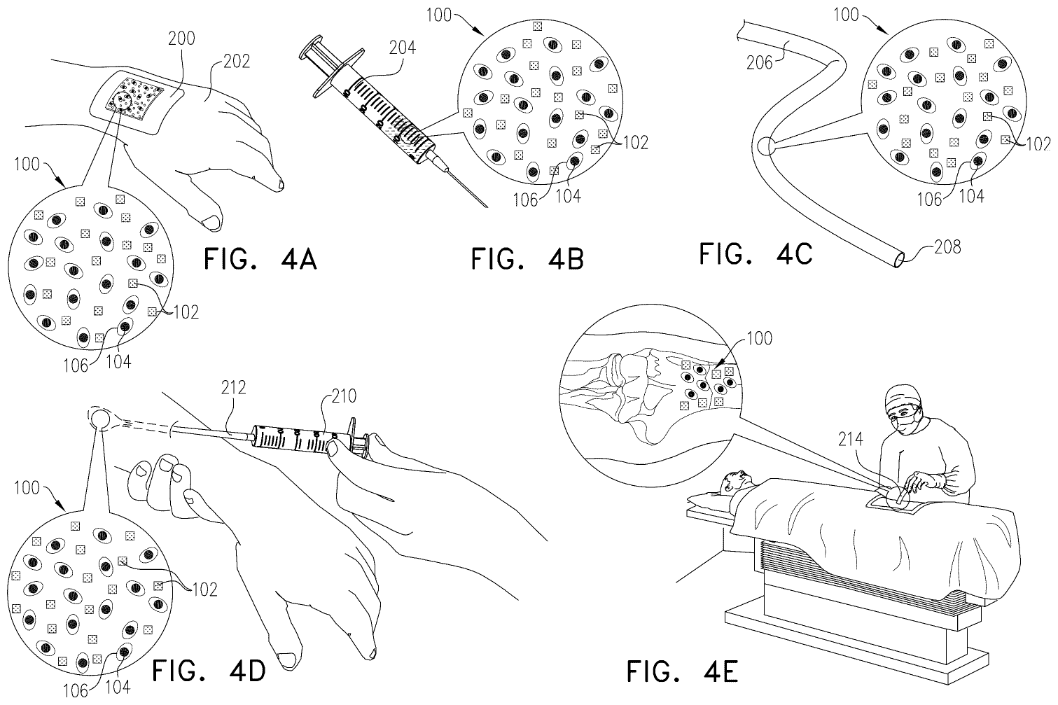

Reference is now additionally made to FIGS. 4A, 4B, 4C, 4D, and 4E, which are schematic illustrations of mechanisms for delivering composition 100 to the anatomical site of the subject, according to some applications of the invention. It is to be appreciated that although FIGS. 4A to 4E illustrate composition 100 as shown in FIG. 1A, the delivery mechanisms illustrated in FIGS. 4A to 4E are similarly useful for delivery of any composition 100 in accordance with the disclosed technology, such compositions 100a, 100b, 100c, 100d, 100e, 100f.

As seen in FIG. 4A, in some implementations, the delivery tool is a patch or bandage 200, applying composition 100 to the skin 202 of the subject, thereby to deliver the composition to the anatomical site. In some implementations, composition 100 serves as an adhesive for adhering patch or bandage 200 to the skin, e.g., as a medical dressing.

Turning to FIG. 4B, in some implementations, the delivery tool is an injection tool, such as syringe 204. The injection tool is used for injecting composition 100 into the anatomical site. In some implementations, the composition can be injected to the anatomical site. For example, the composition may be injected subcutaneously.

Turning to FIG. 4C, in some implementations, the delivery tool is a catheter 206 having a lumen 208. The delivery tool is adapted to be transluminally advanced to the anatomical site. In some implementations, the delivery tool is advanced to the anatomical site with composition 100. In some implementations, the delivery tool is first advanced to the anatomical site, and subsequently composition 100 flows through lumen 208 to the anatomical site.

In FIG. 4D it is shown that, in some implementations, the delivery tool is a subcutaneous delivery tool, such as a syringe 210 in fluid communication with a subcutaneous catheter 212. In such applications, composition 100 can be delivered into the anatomical site subcutaneously.

Turning to FIG. 4E, in some implementations, the delivery tool is a surgical tool 214 for surgically delivering composition 100 to the anatomical site.

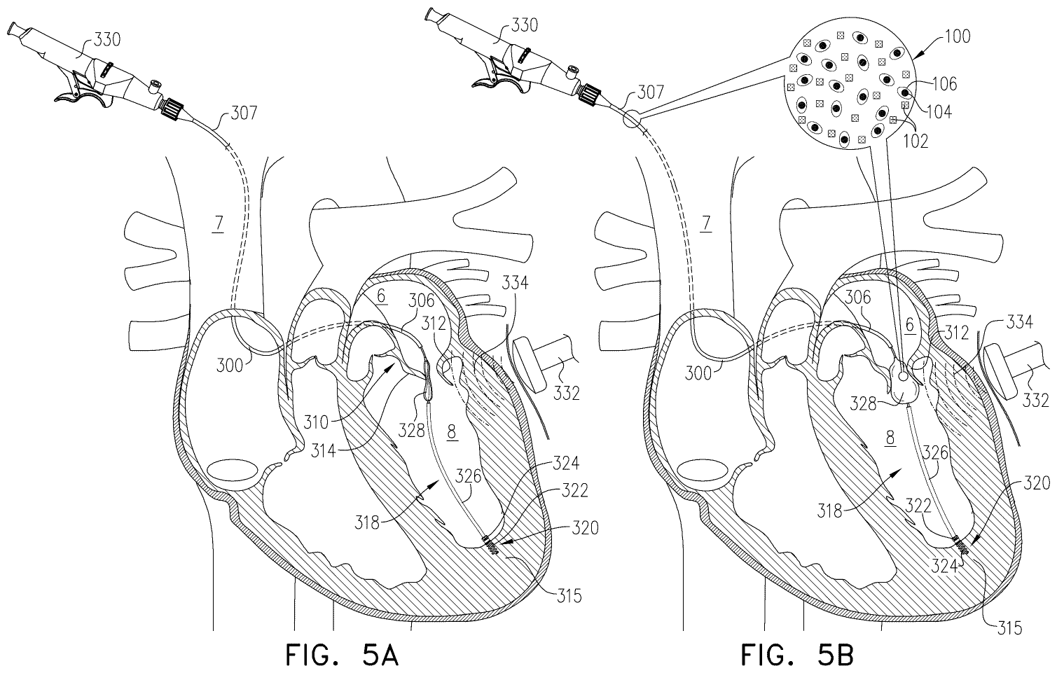

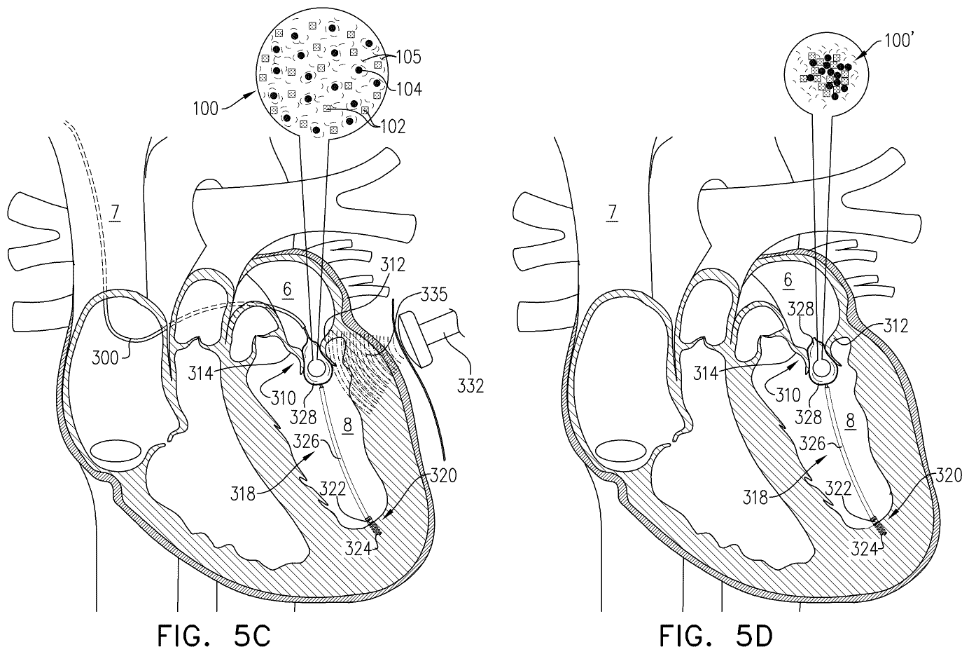

In some implementations, the anatomical site is the heart of the subject, such as a valve of the heart (FIGS. 5A-D, 6A-D, 9A-H). In some implementations, the anatomical site is the skin of the subject, as shown in FIG. 4A. In some implementations, the anatomical site is a bone of the subject, as shown in FIG. 4E.

Returning to FIG. 3, and specifically to steps Va and Vb, composition 100 is shown at an anatomical site 170, which is shown and illustrated as a blood vessel. In the illustrated example, the composition is disposed at the anatomical site within a structure 172. In some implementations, the composition may be delivered to the anatomical site within structure 172, such that the structure functions as a delivery structure. For some other applications, structure 172 may be delivered to the anatomical site in advance of delivery of composition 100 to the anatomical site, such that when the composition is delivered to the anatomical site, it is inserted into the structure already disposed at the anatomical site.

In some implementations, structure 172 can be, or can include, a matrix or a lattice enclosing composition 100. In some implementations, structure 172 can define a cavity 174 accommodating the composition. In some implementations, structure 172 can be a balloon (e.g., a compliant balloon or a non-compliant balloon).

In some implementations, structure 172 can be an implant. In some implementations, composition 100 may be disposed in the implant. Alternatively or in addition, composition 100 may be disposed on a surface of the implant.

As seen at steps Va and Vb, while composition 100 is disposed at anatomical site 170, a de-encapsulation device 176 is used to expose second component 104 to first component 102, by de-encapsulating the second component.

In some implementations, de-encapsulation device 176 comprises an energy applicator 178, applying energy to composition 100, to expose second component 104.

In some implementations, and as shown at step Va, the energy applicator can be an intracorporeal energy applicator, applying energy to composition 100 from within anatomical site 170, or from another location within the body of the subject.

In some implementations, and as shown at step Vb, the energy applicator can be an extracorporeal energy applicator, applying energy to composition 100 from the exterior of the body of the subject.

In some implementations, such as applications in which the first component is also encapsulated (see FIGS. 1D to 1F), at steps Va and Vb, the first component must also be de-encapsulated in order to expose the second component to the first component, and to enable their interaction.

In some implementations, energy applicator 178 comprises an ultrasound transducer (or, as described hereinabove, an ultrasound transceiver). In some implementations, the ultrasound transducer applies high-frequency ultrasound to composition 100. In some implementations, the ultrasound transducer applies high-frequency focused ultrasound (HIFU) to composition 100.

In some implementations in which the first component is also encapsulated, the ultrasound transducer is configured to de-encapsulate the first component and the second component by applying, to composition 100, a single application of ultrasound energy. In such applications, capsules 106 and first capsules 110 may be configured to be sensitive to (e.g., disrupted by) one or more characteristics of ultrasound, e.g., frequency and/or amplitude. In some such applications, capsules 106 and first capsules 110 can have the same physical and/or chemical structure, e.g., may be identical.

In some implementations, energy applicator 178 comprises an ultrasound transducer that can apply to composition 100 ultrasound at a first frequency to de-encapsulate the first component, and apply ultrasound at a second frequency to de-encapsulate the second component. In such applications, capsules 106 and first capsules 110 may be configured to be sensitive to (e.g., disrupted by), different ultrasound frequencies from each other.

In some implementations, even if capsules 106 and 110 are configured to be sensitive to the same frequency of ultrasound, one may require more energy (e.g., a greater ultrasound amplitude) than the other in order to be disrupted sufficiently that its respective component becomes de-encapsulated. For example, ultrasound at a first amplitude may be applied in order to de-encapsulate component 102 but not component 104, and ultrasound at a second, greater, amplitude may be subsequently applied in order to de-encapsulate component 104. In some implementations, this difference may be defined as the different capsules having different resilience.

In some implementations, composition 100 comprises multiple portions of second component 104, each portion being encapsulated in a different capsule type (e.g., having different resilience and/or different frequency sensitivity). Such a composition may allow energy applicator 178 to apply to composition 100 a first energy dose to de-encapsulate a first portion of the second component, and a second energy dose to de-encapsulate a second portion of the second component.

In some implementations, energy applicator 178 comprises an electromagnetic energy source, which applies electromagnetic energy to composition 100 for de-encapsulation of the second component (and, when the first component is encapsulated, of the first component). For example, the electromagnetic energy source may be an ultraviolet light source, an infrared light source, a microwave source, or a radiofrequency (RF) source. In some implementations, the capsule is therefore configured to be sensitive to the electromagnetic energy.

In some implementations, energy applicator 178 comprises one or more electrodes, which apply electrical energy to composition 100 for de-encapsulation of the second component (and, when the first component is encapsulated, of the first component).

In some implementations, the capsules of one component of composition 100 are sensitive to a first energy modality (e.g., ultrasound energy) and the capsules of another component are sensitive to a second energy modality that is qualitatively different from the first energy modality (e.g., electromagnetic energy), thereby advantageously facilitating selective de-encapsulation of each component. Similarly, different modalities may be used to facilitate selective de-encapsulation of portions of a given component.

In some implementations, de-encapsulation device 176 comprises a chemical which, upon being introduced into composition 100, induces a chemical reaction resulting in de-encapsulation of the second component. For example, this chemical may react with capsules 110, or may react with another chemical in the composition to form a product that reacts with capsules 110.

In some implementations, energy applicator 178 is an ultrasound transceiver, and can serve as an imaging device for imaging of anatomical site 170 (and/or composition 100 at the anatomical site) in addition to de-encapsulating one or more components of composition 100. For example, the ultrasound transceiver may be connectable to, or a component of, an imaging system 180 that displays such ultrasound imaging-and optionally also additional information. In some implementations, such imaging is performed prior to de-encapsulation of the second component, e.g., in order to optimally position composition 100, the tool via which it is being delivered, and/or the implant within which it is disposed prior to “activating” the composition by allowing the second component to interact with the first component.

In some implementations, the ultrasound used for such imaging has a different (e.g., lower) frequency to that used for de-encapsulation.

In some implementations, rather than energy applicator 178 serving as an imaging device, a separate imaging device (e.g., a separate ultrasound transceiver) is used for this imaging.

In some implementations, and as illustrated by steps VIa and VIb, subsequent to exposing of the second component to the first component, and optionally allowing time for interaction therebetween, a curing-energy applicator 190 can apply energy to the composition for curing thereof. For example, in some implementations, curing-energy applicator 190 can be an electromagnetic energy source (e.g., an ultraviolet or infrared light source).

In some implementations, curing-energy applicator 190 can be a heating element applying heat to composition 100 (e.g., by contacting the composition and/or by transmitting infrared light to the composition) for curing thereof.

In some implementations, curing-energy applicator 190 can be one or more electrodes, applying electrical energy to composition 100 for curing thereof.

In some implementations, and as shown at step VIa, the curing-energy applicator can be an intracorporeal curing-energy applicator, applying the curing energy to composition 100 from within anatomical site 170, or from another location within the body of the subject.

In some implementations, and as shown at step VIb, the curing-energy applicator can be an extracorporeal curing-energy applicator, applying the curing energy to composition 100 from the exterior of the body of the subject.

Although curing-energy applicator 190 is shown as a discrete device, in some implementations it may be a component of device 176. For example, device 176 may comprise both applicator 178 and 190. Furthermore, in some implementations, the de-encapsulating energy and the curing energy are applied by the same applicator, e.g., a unitary energy applicator may serve as applicator 178 and as applicator 190. For example, such a unitary applicator may be switched between modes that apply qualitatively or quantitively different energies.

In some implementations, and as shown in steps VIa and VIb, exposure of the second component to the first component and/or curing of the composition can yield an at least partially solid body 100′. In some such implementations, body 100′ can form or stabilize a repair structure within the body of the subject, as described hereinbelow.

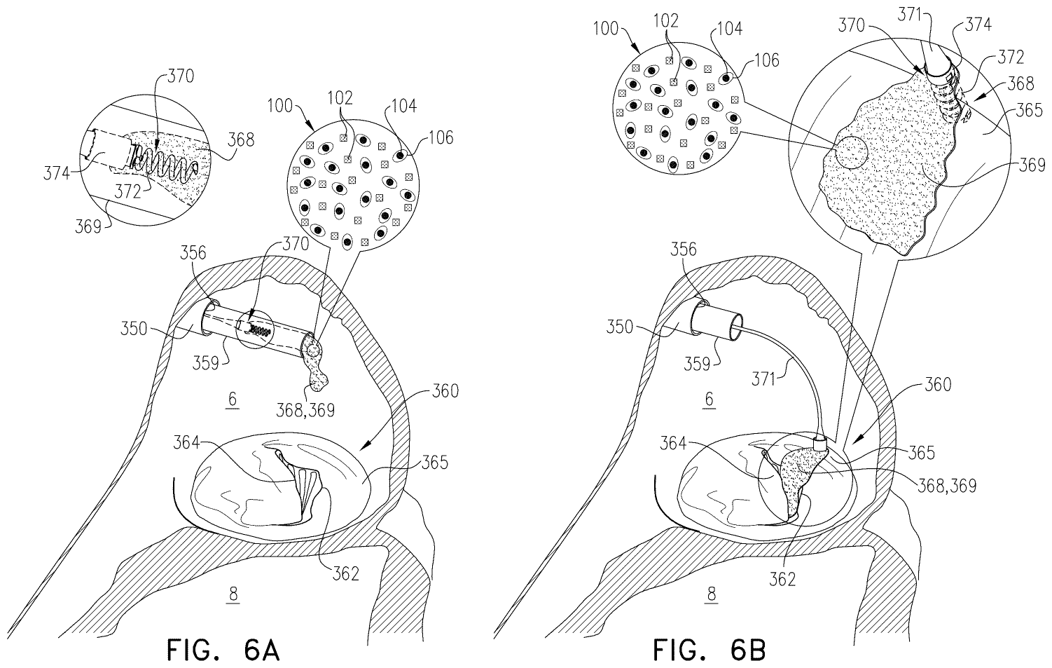

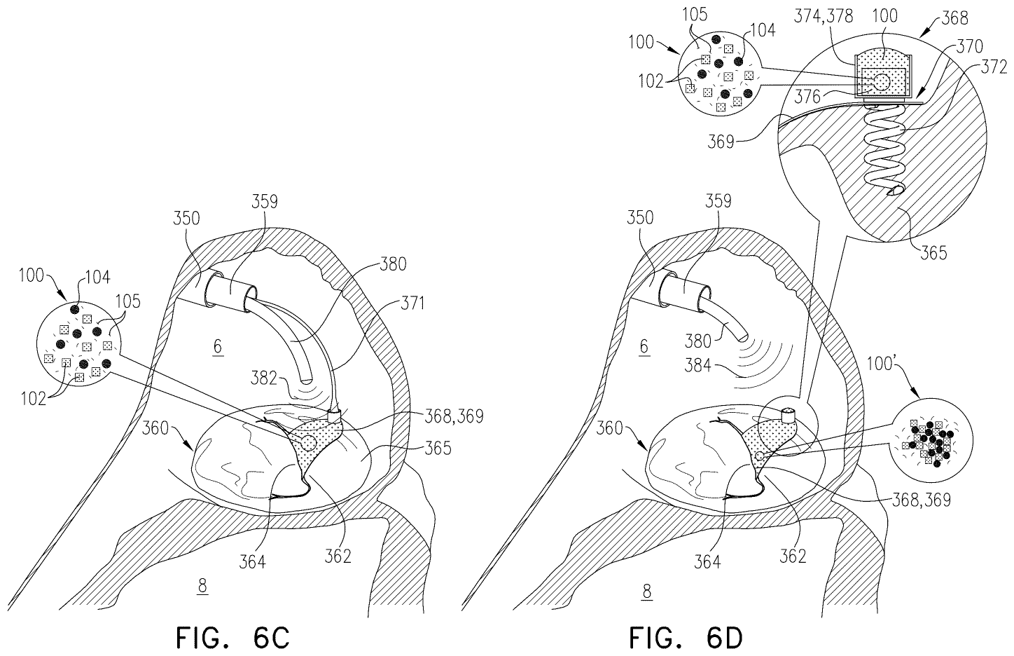

In some implementations, body 100′, or repair structure formed thereof, is used to anchor, or to augment anchoring of, an implant to the anatomical site in the body of the subject, as described hereinbelow with respect to FIGS. 6A-D, 7A-C, 8A-C. In some implementations, body 100′, or a repair structure formed thereof, is used to adhere an implant to the anatomical site.

In some implementations, composition 100 is used to inflate, or shape, an implant at the anatomical site, as described hereinbelow with respect to FIGS. 5A-D and 6A-D. For example, the repair structure may include a coaptation device for improving coaptation of leaflets in a heart valve of the subject, as shown hereinbelow with respect to FIGS. 6A-D, or a spacer for improving function of a heart valve of the subject, as shown hereinbelow with respect to FIGS. 5A to 5D.

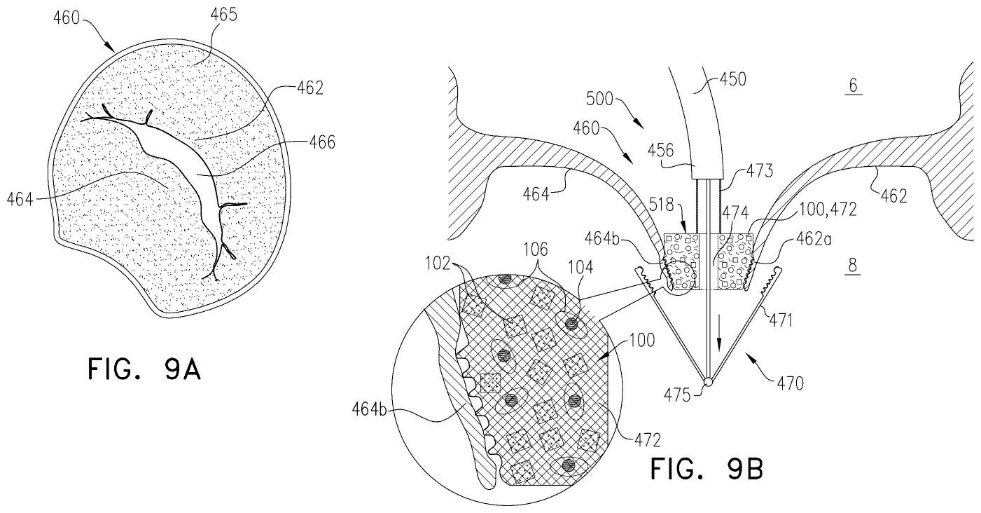

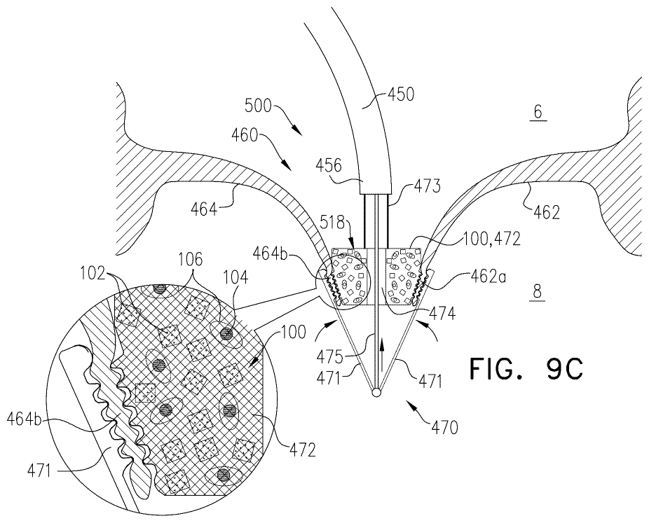

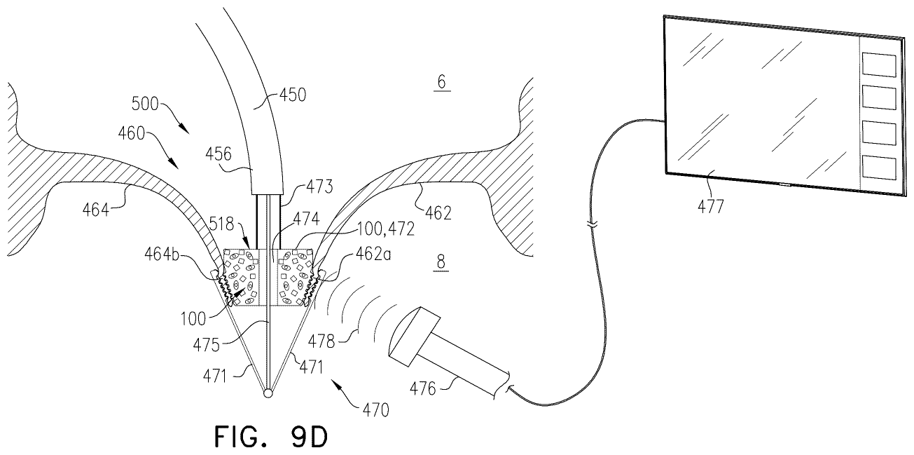

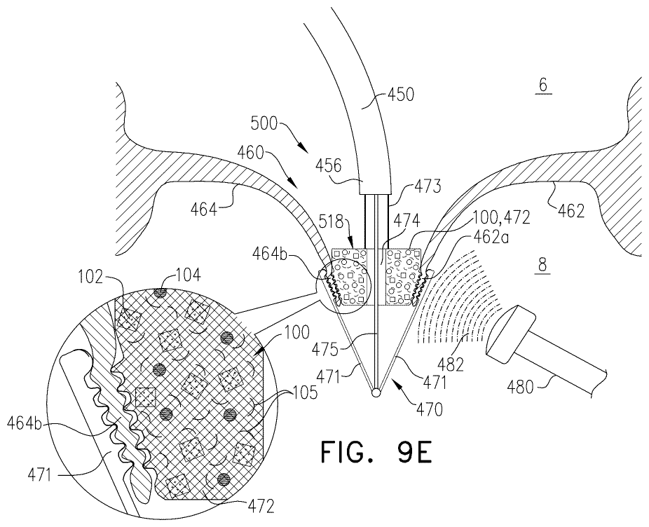

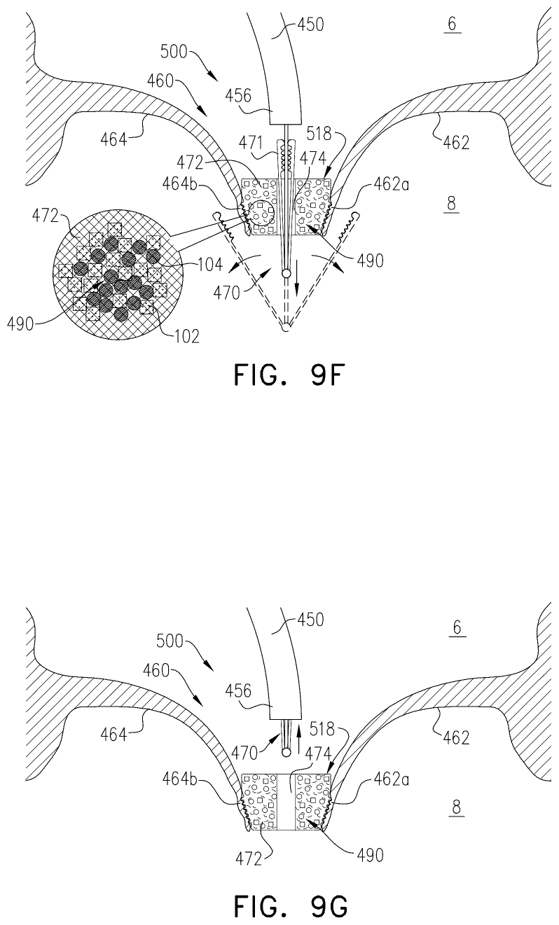

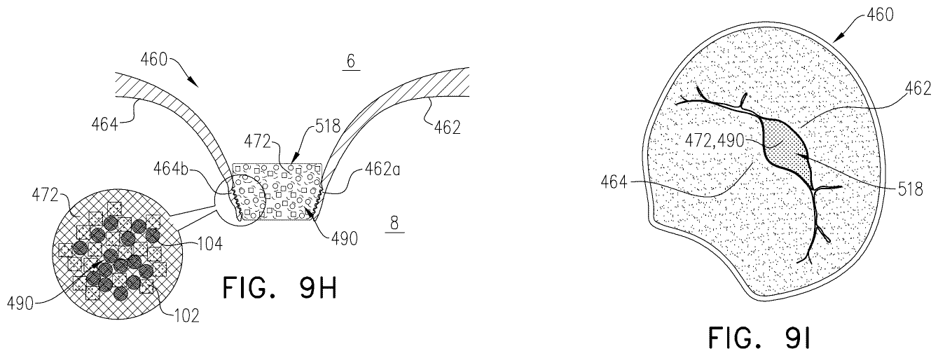

In some implementations, the repair structure is used to reshape tissue at the anatomical site, or to adhere tissue at the anatomical site, for example as shown hereinbelow with respect to FIGS. 9A to 9D.

In some implementations, structure 172 is first placed at the anatomical site, after which composition 100 is added within structure 172. In some such implementations, structure 172 comprises a delivery structure that facilitates delivery of composition 100 by containing the composition as it is placed at the anatomical site. In some such implementations, the delivery structure remains at the anatomical site of the subject subsequent to exposing of the second component to the first component, and/or to curing of the composition, e.g., becomes part of an implant. Alternatively, the delivery structure can be removed from the body of the subject following exposing of the second component to the first component, and/or to curing of the composition. In some implementations, the delivery structure is absorbed into the body of the subject following exposing of the second component to the first component, and/or to curing of the composition.

Reference is now made to FIGS. 5A, 5B, 5C, and 5D, which are schematic illustrations of steps of a method of forming and/or shaping an implant using multi-component compositions 100a, 100b, 100c, 100d, 100e, 100f, according to some applications of the invention. Although FIGS. 5B to 5D illustrate use of composition 100a of FIG. 1A, it is to be appreciated that the method of FIGS. 5A to 5D can similarly be carried out with any of the variations described hereinabove of composition 100.

As seen in FIG. 5A, an implant 318 (e.g., comprising a balloon 328 connected by a tether 326 to an anchor 320) is shown implanted in the heart of a subject. In the example shown, at least part of the implant is positioned downstream of a heart valve 310 (e.g., a mitral valve). The implant is configured to treat valvular regurgitation due to suboptimal coaptation between a first leaflet 312 and a second leaflet 314 of valve 310. In some implementations, implant 318 is delivered to, and implanted at, valve 310 via a longitudinal catheter 300 having a distal part 306 and an extracorporeal proximal part 307. For example, catheter 300, together with implant 318, may have been transluminally advanced toward valve 310 using any method known in the art, for example and as shown, through vena cava 7 and into left ventricle 8 via the interatrial septum and left atrium 6.

In the example shown, implant 318 includes a tissue anchor 320 driven into the tissue of the ventricle, such as into the myocardium 315, for example by a suitable driving tool. The driving tool can extend distally from a lumen of catheter 300. In some implementations, the driving tool can be rotatable, such that rotation of the driving tool screws a portion of tissue anchor 320 into the myocardium 315.

Tissue anchor 320 includes a tissue-engaging element 322, inserted into the tissue of myocardium 315, and a head 324 extending above the tissue at the anatomical site. A tether 326 (e.g., a wire, cable, suture, ribbon, rod, or similar) is coupled to head 324, such that tissue anchor 320 anchors the tether, and balloon 328 is tethered by the tether. In some such implementations, and as shown, the tether's length is selected and/or adjusted such that the balloon is disposed between leaflets 312 and 314.

An ultrasound transceiver 332 can apply ultrasound energy 334 to the anatomical site during and/or after implantation of the implant, to image the implant and the anatomy in order to provide guidance to the operating physician. For example, an ultrasound image can be used to ascertain proper anchoring of tissue anchor 320 and/or positioning of balloon 328. Ultrasound energy 334 can have a wavelength suitable for such imaging of the tissue.

In some implementations, ultrasound transceiver 332 is positioned intracardially. However, in the example shown, it is positioned external to the heart-as represented by the schematic line separating the transceiver from the heart. Such an external position may nonetheless be intracorporeal (e.g., within the esophagus, e.g., transesophageal echocardiography; TEE), or may be extracorporeal (e.g., placed against the skin surface).

In some implementations, balloon 328 is in fluid communication with a lumen of catheter 300. In accordance with some such implementations, FIG. 5B illustrates inflating of balloon 328 by delivery thereinto of composition 100 via catheter 300, Composition 100, including first component 102 and second component 104 encapsulated in capsules 106, is delivered via catheter 300 in order to inflate balloon 328. Inflation balloon 328 causes leaflets 312 and 314 to coapt against the balloon, resulting in proper closing of valve 310, as shown. The ultrasonic imaging described above may also be used to guide inflation of balloon 328, e.g., to ensure that the balloon is inflated to an optimal degree such that leaflets 312 and 314 coapt optimally against the balloon during ventricular systole. Balloon 328 and/or composition 100 may be echogenic, thereby facilitating such imaging.

Turning to FIG. 5C, ultrasound transceiver 332 may then function as a de-encapsulation device, by applying de-encapsulation energy 335 to composition 100 within balloon 328. This de-encapsulation energy may be ultrasound energy 335 that has a higher frequency and/or amplitude than energy 334. Application of ultrasound energy 335 results in rupture/breakdown of capsules 106 (e.g., into fragments 105), which exposes second component 104 to first component 102, and facilitates interaction between the two components. That is, ultrasound transceiver 332 may function both as an imaging device and as a de-encapsulation device. Although in some implementations imaging may continue during de-encapsulation, it is to be noted that these two functions of the ultrasound transceiver are discrete, such that the imaging alone does not cause de-encapsulation, e.g., the physician may choose if and when to induce de-encapsulation.

It is to be appreciated that, in some implementations, another de-encapsulation device may be used.

FIG. 5D shows that following exposure of the components 102 and 104 to each other, they form an at least partially solid body 100′, such as a resin or foam structure, which remains within balloon 328. In some implementations, the techniques described herein mean that final shape of this body (and therefore of balloon 328) is tailored to the heart valve by hemodynamic forces and/or leaflets 312 and 314 pressing against balloon 328, e.g., while composition 100 is still liquid and/or during the period in which it hardens, such that the body is optimal for their coaptation.

As described hereinabove with respect to FIG. 3, in some implementations, body 100′ formed of composition 100 within balloon 328 may be cured (or further cured), by application of energy from a curing energy applicator. Curing body 100′ in balloon 328 may result in further hardening and/or stabilization. In some implementations, a post-de-encapsulation delay (e.g., minutes, hours, or days) may be provided prior to such curing, in order to allow time for formation of the suitably shaped body within balloon 328.

Reference is now made to FIGS. 6A-D, which are schematic illustrations of steps of a method for creating and anchoring an implant 368 using multi-component composition 100, according to some applications of the invention. In some implementations, implant 368 contains composition 100, including first component 102 and second component 104 encapsulated in capsules 106. Although FIGS. 6A to 6D illustrate use of composition 100a, it is to be appreciated that the method of FIGS. 6A-D can similarly be carried out with any variant of composition 100.

In some implementations, implant 368 comprises a wing 369 connected to a tissue anchor 370 (e.g., defining a core 376 and a housing 378, as shown in FIG. 6D). In some such implementations, composition 100 is disposed between layers (e.g., sheets) and/or within pockets defined by wing 369. In some implementations, the wing is formed of a fabric within which composition 100 is embedded or infused.

In some implementations, once implanted, wing 369 extends over leaflet 362, and optionally between leaflets 362 and 364, in a manner that improves coaptation and reduces regurgitation. Implant 368 may thus be considered a leaflet-augmentation implant. In some implementations, implant 368 can be substantially as described and/or serves a similar function to one or more of the implants described in one or more of the following references: International Patent Application No. PCT/US2021/039587 to Chau et al., filed Jun. 29, 2021, which published as WO 2022/006087; and International Patent Application No. PCT/US2022/052834 to Chau et al., filed Dec. 14, 2022

As seen in FIG. 6A, a longitudinal catheter 350 (e.g., having an extracorporeal proximal part similar to that shown in FIG. 5A) is advanced toward an anatomical site of the subject. In the illustrated example, a distal part 356 of the catheter is advanced to an atrium (e.g., a left atrium) of the heart, to be positioned upstream of a heart valve 360 (e.g., a mitral valve) having a first leaflet 362 and a second leaflet 364, surrounded by annulus 365. Distal part 356 is guidable to the anatomical site, such as by being actively steerable itself (e.g., by being operatively coupled by one or more pull wires to the proximal part, such as to a steering controller thereof), or by being passively guided and/or steered (e.g., by extending over or through another steerable element, such as an actively steerable catheter). Catheter 350 is transluminally advanced to the anatomical site using any method known in the art, for example via the vena cava and, if necessary, via the septum separating the atria of the heart, as shown.

In some implementations, and as shown in FIG. 6B, implant 368 is advanced to the anatomical site via a channel or sheath 359 extending longitudinally through catheter 350. In some such implementations, and as shown, tissue anchor 370 is driven into the tissue of annulus 365 (or another tissue adjacent the valve), for example by a suitable driving tool 371, as shown in the inset of FIG. 6B. In some such implementations, and as shown in the inset of FIG. 6B, tissue anchor 370 includes a tissue-engaging element 372, inserted into the tissue, and a head 374 remaining above the tissue. For example, driving tool 371 may extend distally from a lumen of catheter 350. In some implementations, driving tool 371 is rotatable, such that rotation of the driving tool screws a portion of tissue anchor 370 into the tissue.

In accordance with some implementations, FIG. 6C shows use of an intracorporeal de-encapsulation device 380 to apply energy 382 to composition 100 within wing 369. For example, de-encapsulation device 380 may be an ultrasound transducer, e.g., similarly to as described hereinabove, and may be additionally used to guide implantation by ultrasound imaging, e.g., as described for ultrasound transceiver 332, mutatis mutandis. Although de-encapsulation device 380 is shown as intracardial, it may be extracardial, or even extracorporeal, e.g., as described for transceiver 332, mutatis mutandis.

As shown in the inset of FIG. 6C, energy 382 causes breakdown of capsules 106, which exposes second component 104 to first component 102, and facilitates interaction between the two components. In some implementations, interaction of the first and second components within implant 368 can affect the shape and/or flexibility of the implant (e.g., of wing 369), e.g., in a manner that optimizes its leaflet-augmentation functionality. For example, interaction of the first and second components may stiffen the wing of the implant.

In some implementations, the de-encapsulating energy and/or the curing energy is applied gradually and/or in portions, thereby resulting in gradual and/or incremental adjustments to implant 368. Concurrent monitoring of hemodynamics (e.g., via imaging, such as Doppler ultrasound) can thereby allow the implant (e.g., its shape and/or flexibility) to be adjusted in a manner that optimizes its leaflet-augmentation functionality, e.g., until regurgitation is eliminated.

Turning to FIG. 6D, driving tool 371 has been retraced from the anatomical site (e.g., via a lumen of catheter 350) following anchoring of tissue anchor 370 to the tissue. In some implementations, and as shown, de-encapsulation device 380 (and/or a separate curing energy applicator similar to applicator 336 of FIG. 5D or to applicator 190 of FIG. 3) can apply curing energy 384 to composition 100 within implant 368, for curing of the composition.

In some implementations, and as shown, composition 100 is disposed at both wing 369 and at anchor head 374 (e.g., within the anchor head's housing 378). This example is purely illustrative, and that composition 100 may be utilized only in the wing or only in the anchor.

In some implementations, the de-encapsulating energy and/or the curing energy is applied regionally to implant 368, such that the characteristics of given regions (e.g., wing 369 and anchor head 374) can be adjusted. In some such implementations, it might be determined that, in a given case, it would be beneficial to increase the stiffness of the region of the wing closest to anchor 370. For example, curing energy 384 is shown in the lower inset of FIG. 6D as having cured composition 100 on the implant's wing 369 such that the composition forms an at least partially solid body 100′, whereas composition 100 disposed within the anchor's head 374 has been de-encapsulated, yet not fully cured. In this way, composition 100 can increase the wing's stiffness, while forming an adhesive within housing 378 of the tissue anchor head, adhering to core 376, to strengthen the anchoring of the tissue anchor to the annulus. Alternatively, composition 100 within anchor head 374 can be cured following the de-encapsulation step, for example as described hereinabove with respect to FIGS. 3 and 5D.

Following completion of the de-encapsulation (e.g., and curing), catheter 350, together with sheath 359 and de-encapsulation device 380, can be retraced from the anatomical site.

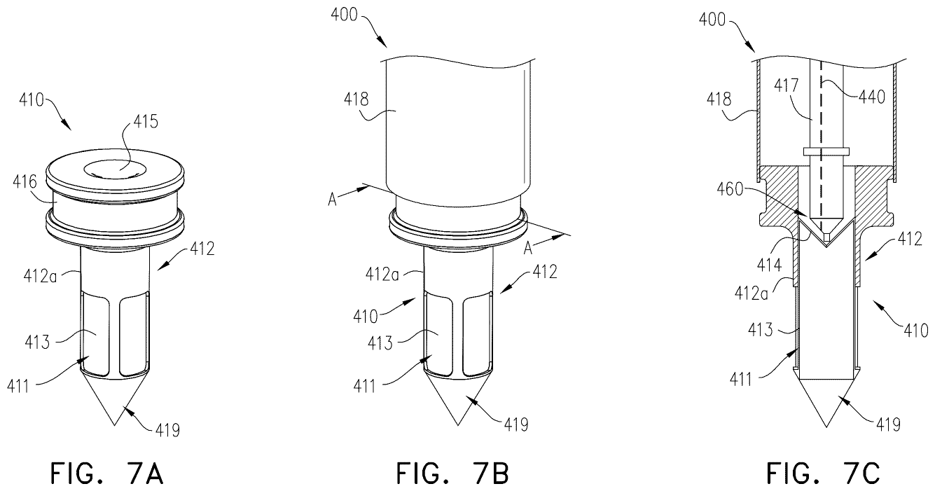

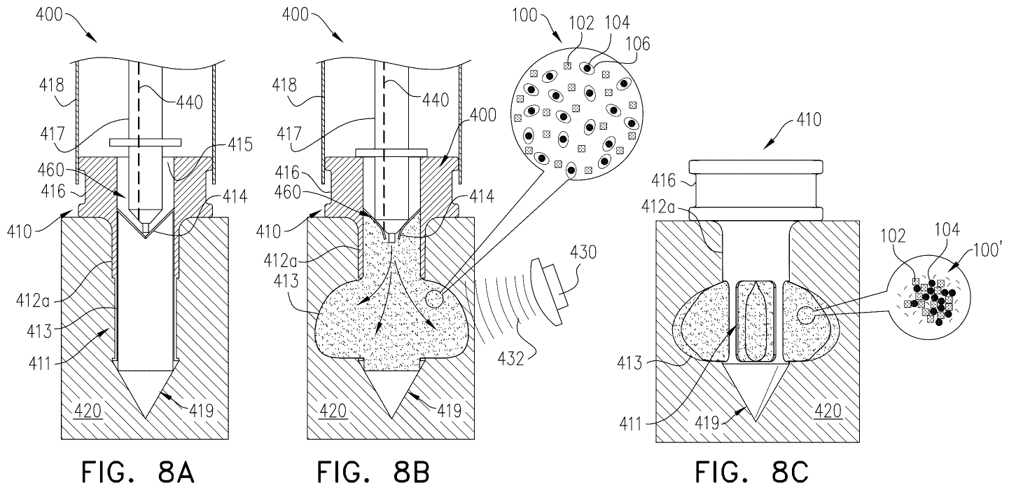

Reference is now made to FIGS. 7A-C, which are schematic illustrations showing a tissue anchor 410 and a system 400 for use with the tissue anchor, and to FIGS. 8A-C, which are schematic illustrations of steps for using the system to implant and anchor the tissue anchor using multi-component compositions 100, according to some applications of the invention. Although FIGS. 8A-C illustrate use of composition 100a of FIG. 1A, it is to be appreciated that the system and method of FIGS. 7A to 8C can similarly be carried out with any one of compositions 100a, 100b, 100c, 100d, 100d, 100e, 100f.

In some implementations, and as shown, anchor 410 comprises a tissue-engaging element 412 and a head 416. Head 416 may comprise an aperture 415 (e.g., a hollow), which may be fitted with an internal closure 414 (see FIG. 7C). Closure 414 may be at a junction of head 416 and tissue-engaging element 412.

In some implementations, and as shown in FIGS. 7B-C, tissue-engaging element 412 may comprise a shaft 412a having a longitudinal axis and a sharp distal tip 419 to facilitate tissue penetration. In some implementations, and as shown in FIG. 7C, shaft 412a is hollow. In some such implementations, and as shown, shaft 412a has a lateral wall defining a series of windows 411 arranged circumferentially around the lateral wall. The series may comprise, for example, two, three, for, five, or six windows. In some implementations, the lateral wall of shaft 412a has only one window. Anchor 410 may further comprise one or more expandable balloons 413, e.g., one balloon, or a set of balloons corresponding to the series of windows, within the shaft of tissue-engaging element 412. Balloon 413 may extend to closure 414, such that the balloon occupies substantially the entire hollow shaft of tissue-engaging element 412.

In some implementations, system 400 comprises, in addition to tissue anchor 410, a delivery tool 418 comprising a balloon expander (e.g., inflator) 417. Balloon expander 417 may be configured to inject, via aperture 415 and through closure 414, a fluid into balloon 413. As discussed hereinbelow, the fluid may be composition 100. Closure 414 may comprise a one-way valve 460 to retain the fluid in the balloon. Delivery tool 418, e.g., balloon expander 417, may further comprise a mechanism to override (e.g., traverse) the valve and withdraw the fluid if needed, e.g., in order to move anchor 410.

In some implementations, and as shown in FIG. 8A, during delivery of tissue anchor 410 to an anatomical site, delivery tool 418 is configured to advance anchor 410 into tissue 420. When the operator is satisfied with the location of anchor 410 in tissue 420, fluid composition 100, may be injected (FIG. 8B) into anchor 410, via balloon expander 417. In some such implementations, and as shown, composition 100 fills balloon(s) 413 and causes the balloon(s) to expand radially, through windows 411 and away from the lateral surface of tissue-engaging element 412, such that the balloon(s) contact, e.g., expand into tissue 420.

In some implementations, and as also seen in FIG. 8B, a de-encapsulation device 430 is used to apply energy 432 to composition 100 within implant balloon(s) 413. This results in breakdown of capsules 106, which exposes second component 104 to first component 102, which facilitates interaction between the two components, as described hereinabove. For example, de-encapsulation device 430 may be an ultrasound transducer, adapted to apply high-frequency ultrasound to composition 100 within balloon(s) 413.

In some implementations, composition 100 (e.g., an epoxy or other resin) is configured to harden after exposure of the first and second components to each other. In some such implementations, to allow for adjustment and repositioning of tissue anchor 410, composition 100 is configured to harden only upon de-encapsulation of the second component and exposure of second component 104 to first component 102. For example, composition 100 may harden only after the operator performs the de-encapsulation step, e.g., after the operator is satisfied with the position and state of the anchor in its inflated state.

In some implementations, following exposure of the first and second components to each other, composition 100 may require application of curing-energy to harden, as described hereinabove with reference to FIG. 6D. In some such implementations, the curing-energy is electromagnetic energy in the ultraviolet range, and an optical fiber 440 (FIGS. 7C, 8A-B) integral to the delivery tool is used to apply UV light to cure composition 100, e.g., when the user is satisfied with the anchor location. In some such implementations, and as shown, optical fiber 440 is coupled to balloon expander 417. For example, optical fiber 440 can access composition 100 via closure 414 or via a window (not shown) near the closure. Alternatively or in addition, the curing-energy may be applied to the composition through tissue 420.

Anchor 410 may be advantageously effective in various applications, e.g., including applications in which the anchor is used to completely traverse thin tissue, and those in which the anchor is anchored into thicker tissue. An advantageous feature of anchor 410 is the ability to expand the balloon laterally beyond the diameter of head 416, such that the anchor may be more resistant to pullout by distributing the force over a greater area of tissue. A still further advantageous feature of anchor 410 is the ability to remove the anchor after removing composition 100 from balloon 413 (s) and/or reinsert the anchor into tissue (e.g., in a minimally traumatic manner) prior to its final securing by curing the composition within the balloon(s).

Reference is now made to FIGS. 9A-I which are schematic illustrations of a system 500 for adhering leaflets 462, 464 of a heart valve 460 to each other using composition 100, according to some applications. Although FIGS. 9A-H illustrate use of composition 100a, it is to be appreciated that the method of FIGS. 9A-H can similarly be carried out with any one of compositions 100a, 100b, 100c, 100d, 100e, 100f.

As seen in FIG. 9A, heart valve 460 (e.g., a mitral valve) has a first leaflet 462 and a second leaflet 464, surrounded by annulus 465. The leaflets of the valve do not optimally coapt during ventricular systole, resulting in regurgitation, e.g., through a gap 466 between the leaflets.

Turning to FIG. 9B, a distal part 456 of a longitudinal catheter 450 is advanced toward heart valve 460 of the subject. In the illustrated example, a distal part 456 of the catheter is advanced to an atrium (e.g., a left atrium 6) of the heart, to be positioned upstream of heart valve 460. Longitudinal catheter 450 also has an extracorporeal proximal part (not shown), similar to that shown in FIG. 5A. Distal part 456 is guidable to the anatomical site, such as by being actively steerable itself (e.g., by being operatively coupled by one or more pull wires to the proximal part, such as to a steering controller thereof), or by being passively guided and/or steered (e.g., by extending over or through another steerable element, such as an actively steerable catheter).

In some implementations, catheter 450 is transluminally advanced to the anatomical site using any method known in the art, for example via the vena cava and, if necessary, via the septum separating the atria of the heart. In some such implementations, and as shown, an implant 518 comprising a grasper 470 and a substrate, e.g., a porous matrix such as a lattice structure 472 on which composition 100 is disposed, are advanced percutaneously through catheter 450. In some such implementations, lattice structure 472 serves as a delivery structure holding composition 100. In some such implementations, lattice structure 472 expands (e.g., automatically self-expands) upon being released from catheter 450.

In some implementations, and as shown in FIG. 9B, lattice structure 472 is advanced out of catheter 450 (e.g., using a pair of grasper connectors 473) such that lattice structure 472 is disposed between leaflets 462, 464, e.g., contacting the leaflets' tips 462a, 464a. In some such implementations, and as shown, grasper 470 extends through a central bore 474 of lattice structure 472. In this way, grasper 470 can be opened, e.g., such that clips pivot away from the grasper's central shaft 475, as shown.

In some implementations, as shown in the inset of FIG. 9B, lattice structure 472 is coated with composition 100 or a variant thereof. For example, the microcapsules of the composition may be bound to the lattice structure, e.g., due to mechanical entrapment, surface chemistry, and/or other surface phenomena. In some implementations, lattice structure 472 and capsules 106 can include tags that interact with each other, to ensure that the capsules remain attached to the lattice during the delivery process.

In some implementations, lattice structure 472 is filled with composition 100 or a variant thereof. For example, lattice structure may contain, or be contained in, a balloon (not shown) that contains the composition. In some implementations, lattice structure 472 can be pre-filled with composition 100 before the lattice structure is delivered to valve 460. Alternatively or in addition, lattice structure 472 can be positioned between leaflets 462 and 464, and then filled with composition 100, e.g., via catheter 450.

In some implementations, lattice structure 472 can be bioabsorbable.

In some implementations, and as shown in FIG. 9C, the grasper's clips 471 are closed upon leaflets 462, 464, e.g., sandwiching the leaflet tips 462a, 464a between the clips and lattice structure 472. In some such implementations, and as shown in the inset of FIG. 9C, surfaces of clips 471 and lattice structure 472 are textured to increase the strength with which the grasper can hold leaflet tips 462a, 464a against lattice structure 472.

In some implementations, and as shown in FIG. 9D, an imaging device 476 is used to image or visualize the anatomical site, for example on a display screen 477, such that the user can ensure that the lattice structure 472 is placed in the correct position. For example, imaging device 476 can be a low frequency ultrasound transceiver, applying low frequency ultrasound waves 478 to valve 460 and to lattice structure 472 for imaging thereof.

In some implementations, imaging device 476 may be delivered to the anatomical site via catheter 450. In some implementations, and as shown, imaging device 476 may be delivered to a region close to the anatomical site using a delivery mechanism other than catheter 450, or may be extracorporeal. In some implementations, lattice structure 472 and/or composition 100 may include a contrast agent, facilitating or aiding in imaging thereof, in the anatomical site.

In some implementations, and as shown in FIG. 9E, a de-encapsulation device 480 is used to apply energy 482 to composition 100 within lattice structure 472. This results in breakdown of the microcapsules of the composition (e.g., into fragments 105), which exposes second component 104 to first component 102, thereby facilitating interaction between the two components. For example, de-encapsulation device 480 may be an ultrasound transducer or transceiver, adapted to apply high-frequency ultrasound to composition 100.

In some implementations, de-encapsulation device 480 and imaging device 476 may be a single ultrasound transducer or transceiver, which applies low frequency ultrasound for imaging of composition 100, and high frequency ultrasound for de-encapsulation of the second component, e.g., as described hereinabove, mutatis mutandis.

In some implementations, interaction of first component 102 with the exposed second component 104 forms an adhesive 490 (FIG. 9F), which adheres leaflets 462 and 464 to lattice structure 472 and/or to each other. In some implementations, following de-encapsulation of second component 104 and formation of adhesive 490 between leaflets 462 and 464, a curing energy applicator (similar to applicator 336 of FIG. 5D or to applicator 190 of FIG. 3) can apply curing energy to composition 100 within lattice structure, for curing of the adhesive.

In some implementations, and as shown in FIG. 9F, following formation, and optional curing, of adhesive 490, catheter 450 disengages lattice structure 472 (e.g., by releasing connectors 473) and grasper 470 releases leaflets 462 and 464 (shown in phantom), such that the grasper can be retracted via bore 474.

In some implementations, and as shown in FIG. 9G, catheter 450 (as well as imaging device 476 and de-encapsulation device 480, if present intracorporeally), are also retracted from the anatomical site.

In some implementations, lattice structure 472 including adhesive 490 remains adhered to leaflets 462 and 464 during (FIG. 9G) and/or following (FIG. 9H) retraction of grasper 470 and catheter 450. In some such implementations, and as shown in FIGS. 9G-H, bore 474 may be a temporary bore that self-seals after removal of grasper 470 from the bore.

In some implementations, and as shown in FIGS. 9H-I, lattice structure 472 and adhesive 490 secure leaflets 462, 464 (e.g., tips 462a, 464a thereof) together, which reduces regurgitation. In some implementations, this may be described as an edge-to-edge repair procedure.

In some implementations, in which lattice structure 472 is biodegradable, over time the lattice structure will biodegrade and will be replaced by tissue growth, keeping the leaflets adhered to each other only by adhesive 490 (FIG. 9I).

In some implementations, if re-intervention is necessary, a physician can cut through adhesive 490, re-separating leaflets 462 and 464 from each other.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

Reference is again made to FIGS. 1A-9I. Although the above disclosure is broadly described in the context of two-part compositions and three-part compositions, it is to be understood that the scope of the disclosure includes other multi-part compositions, e.g., with four or more components, each (e.g., one or more, such as all) of which may be encapsulated.

Example Applications (some non-limiting examples of the concepts herein are recited below):

Example 1. A system for use at an anatomical site of a real or simulated subject, the system comprising an implant that comprises: a porous matrix, and a composition, held in association with the matrix, the composition comprising a mixture that includes: a first component, and a second component, encapsulated in capsules in a manner that prevents its interaction with the first component, the capsules being rupturable to expose the second component to the first component responsively to application of energy to the composition.

Example 2. The system according to example 1, wherein the implant is sterile.

Example 3. The system according to any one of examples 1-2, wherein the capsules comprise polymer capsules.

Example 4. The system according to any one of examples 1-2, wherein the capsules comprise protein capsules.

Example 5. The system according to any one of examples 1-2, wherein the capsules comprise lipid bi-layer capsules.

Example 6. The system according to any one of examples 1-5, wherein the first component is a fluid medium, having suspended therein the capsules.

Example 7. The system according to any one of examples 1-5, wherein the mixture further includes a suspension medium in which the first component and the capsules are suspended.

Example 8. The system according to any one of examples 1-5, wherein the mixture comprises a powder including the first component and the capsules.

Example 9. The system according to any one of examples 1-5, wherein the first component and the second component are two components of a two-part foam.

Example 10. The system according to any one of examples 1-5, wherein the first component and the second component are two components of a two-part polyurethane adhesive.

Example 11. The system according to any one of examples 1-5, wherein the first component and the second component are two components of a two-part resin.

Example 12. The system according to example 11, wherein the first component and the second component are two components of a two-part epoxy resin.

Example 13. The system according to any one of examples 1-12, wherein the mixture further includes a third component.

Example 14. The system according to any one of examples 1-13, further comprising: a delivery tool, configured to transluminally deliver the implant to the anatomical site, and a grasper, configured to hold the implant in contact with tissue at the anatomical site.

Example 15. The system according to example 14, further comprising a de-encapsulation device configured to: be transluminally advanced via the delivery tool to the anatomical site, and expose the second component to the first component by de-encapsulating the second component while the grasper holds the matrix in contact with the tissue at the anatomical site.

Example 16. The system according to example 15, wherein: the grasper is configured to controllably release the matrix; and the composition is configured such that exposing the second component to the first component adheres the matrix to the tissue at the anatomical site, such that the matrix remains adhered to the tissue after the grasper releases the matrix.

Example 17. The system according to any one of examples 15-16, wherein the capsules are second capsules wherein: the first component is encapsulated in first capsules, and the de-encapsulation device is adapted to expose the first component and the second component to each other, by de-encapsulating the first component and the second component while grasper holds the matrix in contact with the tissue at the anatomical site.

Example 18. The system according to example 17, wherein the de-encapsulation device comprises: a first de-encapsulation device adapted to expose the first component by de-encapsulating the first component from the first capsules; and a second de-encapsulation device adapted to expose the second component by de-encapsulating the second component from the second capsules, such that the first component and the second component are exposed to each other.

Example 19. The system according to any one of examples 17-18, wherein the mixture further includes a suspension medium in which both the first capsules and the second capsules are suspended.

Example 20. The system according to any one of examples 17-18, wherein the mixture comprises a powder including the first capsules and the second capsules.

Example 21. The system according to any one of examples 14-20, wherein the mixture further includes a third component.

Example 22. The system according to example 21, wherein the third component comprises a reinforcing component adapted for mechanically reinforcing a repair structure formed by the first component and the second component, subsequent to exposure of the second component by the de-encapsulation device.

Example 23. The system according to example 21, wherein the third component comprises an indicator component adapted to enable visualizing of a location of the mixture within the subject, during or following delivery of the mixture by the delivery tool.

Example 24. The system according to example 21, wherein the third component comprises a surfactant.

Example 25. The system according to example 21, wherein the third component comprises a medicament.

Example 26. The system according to example 25, wherein the medicament is an anti-inflammatory medicament.

Example 27. The system according to any one of examples 25-26, wherein the medicament has tissue-growth-promoting properties.

Example 28. The system according to any one of examples 25-27, wherein the medicament has antimicrobial properties.

Example 29. The system according to example 15, wherein the de-encapsulation device comprises a chemical adapted, upon introduction into the mixture, to carry out a chemical reaction resulting in de-encapsulating of the second component.

Example 30. The system according to example 15, wherein the de-encapsulation device comprises an energy applicator adapted to apply energy to the mixture.

Example 31. The system according to example 30, wherein the energy applicator comprises an electrode adapted to apply electrical energy to the mixture.

Example 32. The system according to example 30, wherein the energy applicator comprises an electromagnetic radiation source adapted to apply electromagnetic energy to the mixture.

Example 33. The system according to example 30, wherein the energy applicator comprises an ultrasound transducer.

Example 34. The system according to example 33, wherein the ultrasound transducer is adapted to apply high frequency ultrasound energy.

Example 35. The system according to example 33, wherein the ultrasound transducer is adapted to apply high-intensity focused ultrasound (HIFU) energy.

Example 36. The system according to any one of examples 33-35, wherein the ultrasound transducer is an intracorporeal ultrasound transducer.

Example 37. The system according to any one of examples 33-35, wherein the ultrasound transducer is an extracorporeal ultrasound transducer.

Example 38. The system according to any one of examples 33-37, wherein the first component of the mixture is also encapsulated, and wherein the ultrasound transducer is adapted to apply ultrasound energy at a single frequency to de-encapsulate the first component and the second component.

Example 39. The system according to any one of examples 33-37, wherein the first component is also encapsulated, and wherein the ultrasound transducer is adapted to apply ultrasound energy at a first frequency to de-encapsulate the first component and at a second frequency to de-encapsulate the second component.

Example 40. The system according to any one of examples 30-39, wherein a first subset of the capsules includes capsules having a first degree of strength, and a second subset of the capsules comprises capsules having a second degree of strength, and the de-encapsulation device is adapted to apply: a first energy dose to the mixture to de-encapsulate the second component from capsules of the first subset, and a second energy dose to the mixture to de-encapsulate the second component from capsules of the second subset, the first and second energy doses being distinct.

Example 41. The system according to any one of examples 15-40, further comprising an imaging system, adapted to image the mixture at the anatomical site.

Example 42. The system according to example 41, wherein the imaging system comprises an ultrasound transducer adapted to apply low-frequency ultrasound.

Example 43. The system according to example 42, wherein the de-encapsulation device and the imaging system comprise a single ultrasound transducer, adapted to apply high-frequency ultrasound to the mixture for de-encapsulation of the second component, and to apply low frequency ultrasound to the mixture for imaging thereof.

Example 44. The system according to any one of examples 1-43, further comprising a curing energy applicator, adapted to apply energy to the mixture for curing thereof subsequently to interaction of the second component with the first component.

Example 45. The system according to example 44, wherein the curing energy applicator comprises an ultraviolet light source adapted to apply to the mixture electromagnetic energy in the ultraviolet region.

Example 46. The system according to example 44, wherein the curing energy applicator comprises an infrared light source adapted to apply to the mixture electromagnetic energy in the infrared region.

Example 47. The system according to example 44, wherein the curing energy applicator comprises a heating element adapted to heat the mixture.

Example 48. The system according to example 44, wherein the curing energy applicator comprises an electrode adapted to apply electrical energy to the mixture.

Example 49. A system for use at an anatomical site of a real or simulated subject, the system comprising: a mixture including a first component and a second component, the second component being encapsulated in frangible capsules that: prevent interaction of the second component with the first component, and are rupturable to expose the second component to the first component responsively to application of energy to the frangible capsules; and a delivery tool adapted to deliver the mixture toward the anatomical site.

Example 50. The system according to example 49, at least one of the mixture, and the delivery tool is sterile.

Example 51. The system according to any one of examples 49-50, wherein the frangible capsules comprise frangible polymer capsules.

Example 52. The system according to any one of examples 49-50, wherein the frangible capsules comprise frangible protein capsules.

Example 53. The system according to any one of examples 49-50, wherein the frangible capsules comprise frangible lipid bi-layer capsules.

Example 54. The system according to any one of examples 49-53, wherein the first component is a fluid medium, having suspended therein the frangible capsules.

Example 55. The system according to any one of examples 49-53, wherein the mixture further includes a suspension medium in which the first component and the frangible capsules are suspended.

Example 56. The system according to any one of examples 49-53, wherein the mixture comprises a powder including the first component and the frangible capsules.

Example 57. The system according to any one of examples 49-53, wherein the first component and the second component are two components of a two-part foam.

Example 58. The system according to any one of examples 49-53, wherein the first component and the second component are two components of a two-part polyurethane adhesive.

Example 59. The system according to any one of examples 49-53, wherein the first component and the second component are two components of a two-part resin.

Example 60. The system according to example 59, wherein the first component and the second component are two components of a two-part epoxy resin.

Example 61. The system according to any one of examples 49-60, further comprising a de-encapsulation device adapted to rupture the frangible capsules by applying energy to the frangible capsules.

Example 62. The system according to example 61, wherein: the frangible capsules are second frangible capsules, the first component is encapsulated in first frangible capsules, and the de-encapsulation device is adapted to expose the second component to the first component by applying energy to the first frangible capsules and to the second frangible capsules.

Example 63. The system according to example 62, wherein the mixture further includes a suspension medium in which both the first frangible capsules and the second frangible capsules are suspended.

Example 64. The system according to example 62, wherein the mixture comprises a powder including the first frangible capsules and the second frangible capsules.

Example 65. The system according to any one of examples 49-64, wherein the mixture further includes a third component.

Example 66. The system according to example 65, wherein the third component comprises a reinforcing component adapted for mechanically reinforcing a repair structure formed by the first component and the second component, subsequent to exposure of the second component by the de-encapsulation device.

Example 67. The system according to example 65, wherein the third component comprises an indicator component adapted to enable visualizing of a location of the mixture within the subject, during or following delivery of the mixture by the delivery tool.

Example 68. The system according to example 65, wherein the third component comprises a surfactant.

Example 69. The system according to example 65, wherein the third component comprises a medicament.

Example 70. The system according to example 69, wherein the medicament is an anti-inflammatory medicament.

Example 71. The system according to any one of examples 69-70, wherein the medicament has tissue-growth-promoting properties.

Example 72. The system according to any one of examples 69-71, wherein the medicament has antimicrobial properties.

Example 73. The system according to any one of examples 49-72, wherein the delivery tool comprises a catheter, adapted to be transluminally advanced, with the mixture, to the anatomical site.

Example 74. The system according to example 73, wherein the catheter defines a lumen, the catheter being adapted to have the mixture advanced to the anatomical site via the lumen.

Example 75. The system according to example 73, further comprising a delivery structure containing the mixture, the delivery structure adapted to be transluminally advanced to the anatomical site via the catheter, with the mixture disposed therein.

Example 76. The system according to example 75, wherein the delivery structure includes a cavity accommodating the mixture.

Example 77. The system according to example 76, wherein the delivery structure comprises a balloon.

Example 78. The system according to example 76, wherein the delivery structure comprises a lattice structure holding the mixture.

Example 79. The system according to example 78, wherein the lattice structure is configured to be transluminally advanced via the catheter, and to automatically self-expand upon release from the catheter.

Example 80. The system according to example 78, wherein the lattice structure is configured to remain at the anatomical site, subsequent to exposure of the second component by the de-encapsulation device.

Example 81. The system according to example 78, wherein: the system further comprises a grasper, configured to be advanced transluminally via the delivery tool, and to hold the lattice structure in contact with tissue of the anatomical site; and the de-encapsulation device is configured to expose the second component to the first component by de-encapsulating the second component while the grasper holds the lattice structure in contact with tissue of the anatomical site.

Example 82. The system according to example 81, wherein the grasper is configured to be withdrawn from the subject via the delivery tool, subsequent to exposure of the second component by the de-encapsulation device.

Example 83. The system according to example 81, configured to de-encapsulate the second component while the grasper holds the lattice structure in contact with tissue of the anatomical site, such that the lattice structure adheres to tissue of the anatomical site.

Example 84. The system according to example 83, wherein: the anatomical site is at a native valve of a real or simulated heart of the subject; the grasper is configured to hold the lattice structure in contact with leaflets of the native valve; and the lattice structure is configured to remain adhered to leaflets of the native valve, subsequent to exposure of the second component by the de-encapsulation device.

Example 85. The system according to example 75, wherein the delivery structure comprises an implant adapted to be implanted at the anatomical site such that the delivery structure remains within the subject subsequently to exposure of the second component to the first component.

Example 86. The system according to example 75, wherein the delivery structure is absorbed into the subject subsequently to exposure of the second component to the first component.

Example 87. The system according to example 75, wherein the delivery structure is adapted to be removed from the subject subsequently to exposure of the second component to the first component.

Example 88. The system according to any one of examples 49-72, wherein the delivery tool comprises a tool for applying the mixture to skin of the subject.

Example 89. The system according to any one of examples 49-72, wherein the delivery tool comprises a tool for surgically delivering the mixture to the anatomical site.

Example 90. The system according to any one of examples 49-72, wherein the delivery tool comprises an injection tool for injecting the mixture into the subject.

Example 91. The system according to any one of examples 49-72, wherein the delivery tool comprises a subcutaneous delivery tool.

Example 92. The system according to any one of examples 49-91, wherein the de-encapsulation device comprises a chemical adapted, upon introduction into the mixture, to carry out a chemical reaction resulting in de-encapsulating of the second component.

Example 93. The system according to any one of examples 49-91, wherein the de-encapsulation device comprises an energy applicator adapted to apply energy to the mixture.

Example 94. The system according to example 93, wherein the energy applicator comprises an electrode adapted to apply electrical energy to the mixture.

Example 95. The system according to example 93, wherein the energy applicator comprises an electromagnetic radiation source adapted to apply electromagnetic energy to the mixture.

Example 96. The system according to example 93, wherein the energy applicator comprises an ultrasound transducer.

Example 97. The system according to example 96, wherein the ultrasound transducer is adapted to apply high frequency ultrasound energy.

Example 98. The system according to example 96, wherein the ultrasound transducer is adapted to apply high-intensity focused ultrasound (HIFU) energy.

Example 99. The system according to any one of examples 96-98, wherein the ultrasound transducer is an intracorporeal ultrasound transducer.

Example 100. The system according to any one of examples 96-98, wherein the ultrasound transducer is an extracorporeal ultrasound transducer.

Example 101. The system according to any one of examples 96-100, wherein the first component of the mixture is also encapsulated, and wherein the ultrasound transducer is adapted to apply ultrasound energy at a single frequency to de-encapsulate the first component and the second component.

Example 102. The system according to any one of examples 96-100, wherein the first component is also encapsulated, and wherein the ultrasound transducer is adapted to apply ultrasound energy at a first frequency to de-encapsulate the first component and at a second frequency to de-encapsulate the second component.

Example 103. The system according to any one of examples 93-102, wherein a first subset of the frangible capsules includes frangible capsules having a first degree of strength, and a second subset of the frangible capsules comprises frangible capsules having a second degree of strength, and the de-encapsulation device is adapted to apply: a first energy dose to the mixture to de-encapsulate the second component from frangible capsules of the first subset, and a second energy dose to the mixture to de-encapsulate the second component from frangible capsules of the second subset, the first and second energy doses being distinct.

Example 104. The system according to any one of examples 49-103, further comprising an imaging system, adapted to image the mixture at the anatomical site.

Example 105. The system according to example 104, wherein the imaging system comprises an ultrasound transducer adapted to apply low-frequency ultrasound.

Example 106. The system according to example 105, wherein the de-encapsulation device and the imaging system comprise a single ultrasound transducer, adapted to apply high-frequency ultrasound to the mixture for de-encapsulation of the second component, and to apply low frequency ultrasound to the mixture for imaging thereof.

Example 107. The system according to any one of examples 49-106, further comprising a curing energy applicator, adapted to apply energy to the mixture for curing thereof subsequently to exposure of the second component to the first component.

Example 108. The system according to example 107, wherein the curing energy applicator comprises an ultraviolet light source adapted to apply to the mixture electromagnetic energy in the ultraviolet region.

Example 109. The system according to example 107, wherein the curing energy applicator comprises an infrared light source adapted to apply to the mixture electromagnetic energy in the infrared region.

Example 110. The system according to example 107, wherein the curing energy applicator comprises a heating element adapted to heat the mixture.

Example 111. The system according to example 107, wherein the curing energy applicator comprises an electrode adapted to apply electrical energy to the mixture.

Example 112. A method, comprising: delivering, to an anatomical site of a real or simulated subject, a mixture including: a first component, and a second component, encapsulated in a manner that prevents its interaction with the first component; and while the mixture remains at the anatomical site, exposing the second component to the first component by de-encapsulating the second component.

Example 113. The method according to example 112, further comprising sterilizing the mixture.

Example 114. The method according to any one of examples 112-113, wherein the first component is a fluid medium, having suspended therein the encapsulated second component.

Example 115. The method according to any one of examples 112-113, wherein the mixture further includes a suspension medium in which the first component and the encapsulated second component are suspended, and wherein delivering the mixture comprises delivering the mixture that further includes the suspension medium.

Example 116. The method according to any one of examples 112-113, wherein the mixture comprises a powder including the first component and the encapsulated second component, and wherein delivering the mixture comprises delivering the powder.

Example 117. The method according to any one of examples 112-116, wherein: the first component is encapsulated in first capsules, the encapsulation of the second component being encapsulation in second capsules, and exposing the second component to the first component comprises de-encapsulating both the first component and the second component.

Example 118. The method according to any one of examples 112-117, wherein the mixture further includes a third component, and wherein delivering the mixture comprises delivering the mixture that further includes the third component.

Example 119. The method according to example 118, wherein the third component comprises a reinforcing component, the method further comprising using the reinforcing component, mechanically reinforcing a repair structure formed by the first component and the second component, subsequent to exposing of the second component.

Example 120. The method according to example 118, wherein the third component comprises an indicator component, the method further comprising visualizing a location of the mixture within the subject, during or following delivering of the mixture, by visualizing of the indicator component.

Example 121. The method according to example 118, wherein the third component comprises a surfactant, and wherein delivering the mixture comprises delivering the mixture that further includes the surfactant.

Example 122. The method according to example 118, wherein the third component comprises a medicament, and wherein delivering the mixture comprises delivering the mixture that further includes the medicament.

Example 123. The method according to example 122, wherein the medicament is an anti-inflammatory medicament, and wherein delivering the mixture comprises delivering the mixture that further includes the anti-inflammatory medicament.

Example 124. The method according to any one of examples 122-123, wherein the medicament is a tissue-growth-promoting medicament, and wherein delivering the mixture comprises delivering the mixture that further includes the tissue-growth-promoting medicament.

Example 125. The method according to any one of examples 122-124, wherein the medicament is an antimicrobial medicament, and wherein delivering the mixture comprises delivering the mixture that further includes the antimicrobial medicament.

Example 126. The method according to any one of examples 112-125, wherein delivering comprises applying the mixture to skin of the subject.

Example 127. The method according to any one of examples 112-125, wherein delivering comprises surgically delivering the mixture to the anatomical site.

Example 128. The method according to any one of examples 112-125, wherein delivering comprises subcutaneously delivering the mixture to the anatomical site.

Example 129. The method according to any one of examples 112-125, wherein delivering comprises transluminally advancing the mixture to the anatomical site.

Example 130. The method according to example 129, wherein transluminally advancing comprises transluminally advancing the mixture to the anatomical site within a delivery structure containing the mixture.

Example 131. The method according to example 130, further comprising removing the delivery structure from the subject subsequently to exposing the second component to the first component.

Example 132. The method according to example 130, wherein advancing the delivery structure to the anatomical site comprises implanting the delivery structure at the anatomical site such that the delivery structure remains within the subject subsequently to exposing the second component to the first component.

Example 133. The method according to example 132 wherein: transluminally advancing comprises transluminally advancing the mixture to the anatomical site via a catheter; and the method further comprises, subsequently to exposing the second component to the first component, removing the catheter from the subject such that the delivery structure remains within the subject.

Example 134. The method according to any one of examples 112-125, wherein delivering comprises injecting the mixture into the subject.

Example 135. The method according to example 134, wherein injecting comprises injecting the mixture to the anatomical site.

Example 136. The method according to example 134, wherein injecting comprises injecting the mixture into a lumen of the subject having a fluid flow therethrough, at a location remote from the anatomical site, the mixture flowing with the fluid via the lumen to the anatomical site.

Example 137. The method according to any one of examples 112-136, wherein delivering the mixture to the anatomical site comprises delivering the mixture to a real or simulated heart of the subject.

Example 138. The method according to example 137, wherein delivering the mixture to the anatomical site comprises delivering the mixture to a valve of the heart of the subject.

Example 139. The method according to any one of examples 112-138, wherein de-encapsulating comprises introducing into the mixture a chemical which carries out a chemical reaction resulting in de-encapsulating of the second component.

Example 140. The method according to any one of examples 112-138, wherein de-encapsulating comprises applying energy to the mixture.

Example 141. The method according to example 140, wherein applying the energy comprises applying electrical energy to the mixture.

Example 142. The method according to example 140, wherein applying the energy comprises applying electromagnetic energy to the mixture.

Example 143. The method according to example 140, wherein applying the energy comprises applying ultrasound energy to the mixture.

Example 144. The method according to example 143, wherein applying ultrasound energy comprises applying high-frequency ultrasound energy.

Example 145. The method according to example 143, wherein applying ultrasound energy comprises applying high-intensity focused ultrasound (HIFU) energy.

Example 146. The method according to any one of examples 143-145, wherein applying ultrasound energy comprises applying ultrasound energy from an ultrasound transducer disposed within the subject.

Example 147. The method according to any one of examples 143-145, wherein applying ultrasound energy comprises applying ultrasound energy from an ultrasound transducer exterior to the subject.

Example 148. The method according to any one of examples 143-147, wherein the first component is also encapsulated, and wherein applying ultrasound energy comprises applying ultrasound energy at a single frequency to de-encapsulate the first component and the second component.

Example 149. The method according to any one of examples 143-147, wherein the first component is also encapsulated, and wherein applying ultrasound energy comprises applying ultrasound energy at a first frequency to de-encapsulate the first component and at a second frequency to de-encapsulate the second component.

Example 150. The method according to any one of examples 112-139, further comprising mixing the first component and the encapsulated second component to form the mixture.

Example 151. The method according to any one of examples 112-150, further comprising encapsulating the second component within capsules.

Example 152. The method according to example 151, wherein encapsulating comprises encapsulating the second component within polymer capsules.

Example 153. The method according to example 151, wherein encapsulating comprises encapsulating the second component within protein capsules.

Example 154. The method according to example 151, wherein encapsulating comprises encapsulating the second component within lipid bilayer capsules.

Example 155. The method according to any one of examples 151-154, wherein encapsulating comprises encapsulating a first portion of the second component in capsules having a first degree of strength, and encapsulating a second portion of the second component in capsules having a second degree of strength, wherein de-encapsulating comprises applying energy to the mixture to de-encapsulate the second component, and wherein the first and second degrees of strength require distinct energy doses for de-encapsulation.

Example 156. The method according to any one of examples 112-138, further comprising, prior to de-encapsulating, imaging the mixture at the anatomical site.

Example 157. The method according to example 156, wherein imaging the mixture at the anatomical site comprises applying imaging ultrasound energy to the mixture at the anatomical site.

Example 158. The method according to example 157, wherein de-encapsulating comprises applying de-encapsulating ultrasound energy to the mixture, the de-encapsulating ultrasound energy having a frequency that is higher than that of the imaging ultrasound energy.

Example 159. The method according to example 158, wherein applying the imaging ultrasound energy and applying the de-encapsulating ultrasound energy comprise applying the imaging ultrasound energy and applying the de-encapsulating ultrasound energy using a single ultrasound transducer.

Example 160. The method according to example 158, wherein applying the de-encapsulating ultrasound energy comprises applying the de-encapsulating ultrasound energy responsively to imaging the mixture at the anatomical site.

Example 161. The method according to any one of examples 112-160, further comprising, following exposing the second component to the first component, curing the mixture by applying curing-energy to the mixture.

Example 162. The method according to example 161, wherein applying curing-energy comprises illuminating the mixture with ultraviolet light.

Example 163. The method according to example 161, wherein applying curing-energy comprises illuminating the mixture with infrared light.

Example 164. The method according to example 161, wherein applying curing-energy comprises heating the mixture.

Example 165. The method according to example 161, wherein applying curing-energy comprises applying electrical energy to the mixture.

Example 166. The method according to any one of examples 112-165, wherein the first component and the second component are two components of a two-part resin, and delivering the mixture comprises delivering the two components of the two-part resin.

Example 167. The method according to any one of examples 112-165, wherein the first component and the second component are two components of a two-part foam, and delivering the mixture comprises delivering the two components of the two-part foam.

Example 168. The method according to any one of examples 112-165, wherein the first component and the second component are two components of a two-part polyurethane adhesive, and delivering the mixture comprises delivering the two components of the two-part polyurethane adhesive.

Example 169. The method according to any one of examples 112-168, wherein: exposing the second component to the first component forms a repair structure, and the method further comprising using the repair structure, anchoring an implant at the anatomical site.

Example 170. The method according to any one of examples 112-168, wherein: exposing the second component to the first component forms a repair structure, and the method further comprising using the repair structure, augmenting anchoring of an implant at the anatomical site.

Example 171. The method according to any one of examples 112-168, wherein: exposing the second component to the first component forms a repair structure, and the method further comprising using the repair structure, reshaping tissue at the anatomical site.

Example 172. The method according to any one of examples 112-168, wherein: exposing the second component to the first component forms a repair structure, and the method further comprising using the repair structure, adhering tissue at the anatomical site.

Example 173. The method according to any one of examples 112-168, wherein; exposing the second component to the first component forms a repair structure, and the method further comprising using the repair structure, adhering an implant to the anatomical site.

Example 174. The method according to any one of examples 112-168, wherein exposing the second component to the first component comprises forming a repair structure within an implant at the anatomical site, the repair structure inflating the implant at the anatomical site.

Example 175. The method according to example 174, wherein forming the repair structure comprises inflating a coaptation device improving coaptation of leaflets of a real or simulated heart valve of the subject.

Example 176. The method according to example 174, wherein forming the repair structure comprises inflating a spacer improving function of a real or simulated heart valve of the subject.

Example 177. A method, comprising: delivering, to an anatomical site of a simulated subject, a mixture including: a first component, and a second component, encapsulated in a manner that prevents its interaction with the first component; and while the mixture remains at the anatomical site, exposing the second component to the first component by de-encapsulating the second component.

Example 178. A system for use at an anatomical site of a real or simulated subject, the system comprising: a mixture including a first component and a second component, the second component being encapsulated in capsules in a manner that prevents its interaction with the first component; a delivery tool adapted to deliver the mixture toward the anatomical site; and a de-encapsulation device adapted to expose the second component to the first component by de-encapsulating the second component.

Example 179. The system according to example 178, wherein at least one of the mixture, the delivery tool and the de-encapsulation device is sterile.

Example 180. The system according to any one of examples 178-179, further comprising a mixing device adapted to mix the first component and the encapsulated second component to form the mixture.

Example 181. The system according to any one of examples 178-180, wherein the capsules comprise polymer capsules.

Example 182. The system according to any one of examples 178-180, wherein the capsules comprise protein capsules.

Example 183. The system according to any one of examples 178-180, wherein the capsules comprise lipid bi-layer capsules.

Example 184. The system according to any one of examples 178-183, wherein the first component is a fluid medium, having suspended therein the capsules.

Example 185. The system according to any one of examples 178-183, wherein the mixture further includes a suspension medium in which the first component and the capsules are suspended.

Example 186. The system according to any one of examples 178-183, wherein the mixture comprises a powder including the first component and the capsules.

Example 187. The system according to any one of examples 178-183, wherein the first component and the second component are two components of a two-part foam.

Example 188. The system according to any one of examples 178-183, wherein the first component and the second component are two components of a two-part polyurethane adhesive.

Example 189. The system according to any one of examples 178-183, wherein the first component and the second component are two components of a two-part resin.

Example 190. The system according to example 189, wherein the first component and the second component are two components of a two-part epoxy resin.

Example 191. The system according to any one of examples 178-183, wherein the capsules are second capsules, and wherein the first component is encapsulated in first capsules.

Example 192. The system according to example 191, wherein the de-encapsulation device is adapted to expose the first component and the second component to each other, by de-encapsulating the first component and the second component.

Example 193. The system according to example 192, wherein the de-encapsulation device comprises: a first de-encapsulation device adapted to expose the first component by de-encapsulating the first component from the first capsules; and a second de-encapsulation device adapted to expose the second component by de-encapsulating the second component from the second capsules, such that the first component and the second component are exposed to each other.

Example 194. The system according to any one of examples 191-193, wherein the mixture further includes a suspension medium in which both the first capsules and the second capsules are suspended.

Example 195. The system according to any one of examples 191-193, wherein the mixture comprises a powder including the first capsules and the second capsules.

Example 196. The system according to any one of examples 178-195, wherein the mixture further includes a third component.

Example 197. The system according to example 196, wherein the third component comprises a reinforcing component adapted for mechanically reinforcing a repair structure formed by the first component and the second component, subsequent to exposure of the second component by the de-encapsulation device.

Example 198. The system according to example 196, wherein the third component comprises an indicator component adapted to enable visualizing of a location of the mixture within the subject, during or following delivery of the mixture by the delivery tool.

Example 199. The system according to example 196, wherein the third component comprises a surfactant.

Example 200. The system according to example 196, wherein the third component comprises a medicament.

Example 201. The system according to example 200, wherein the medicament is an anti-inflammatory medicament.

Example 202. The system according to any one of examples 200-201, wherein the medicament has tissue-growth-promoting properties.

Example 203. The system according to any one of examples 200-202, wherein the medicament has antimicrobial properties.

Example 204. The system according to any one of examples 178-203, wherein the delivery tool comprises a catheter, adapted to be transluminally advanced, with the mixture, to the anatomical site.

Example 205. The system according to example 204, wherein the catheter defines a lumen, the catheter being adapted to have the mixture advanced to the anatomical site via the lumen.

Example 206. The system according to example 204, further comprising a delivery structure containing the mixture, the delivery structure adapted to be transluminally advanced to the anatomical site via the catheter, with the mixture disposed therein.

Example 207. The system according to example 206, wherein the delivery structure comprises a lattice structure holding the mixture.

Example 208. The system according to example 207, wherein the lattice structure is configured to be transluminally advanced via the catheter, and to automatically self-expand upon release from the catheter.

Example 209. The system according to example 207, wherein the lattice structure is configured to remain at the anatomical site, subsequent to exposure of the second component by the de-encapsulation device.

Example 210. The system according to example 207, wherein: the system further comprises a grasper, configured to be advanced transluminally via the delivery tool, and to hold the lattice structure in contact with tissue of the anatomical site; and the de-encapsulation device is configured to expose the second component to the first component by de-encapsulating the second component while the grasper holds the lattice structure in contact with tissue of the anatomical site.

Example 211. The system according to example 210, wherein the grasper is configured to be withdrawn from the subject via the delivery tool, subsequent to exposure of the second component by the de-encapsulation device.

Example 212. The system according to example 210, configured to de-encapsulate the second component while the grasper holds the lattice structure in contact with tissue of the anatomical site such that the lattice structure adheres to tissue of the anatomical site.

Example 213. The system according to example 212, wherein: the anatomical site is at a native valve of a real or simulated heart of the subject; the grasper is configured to hold the lattice structure in contact with leaflets of the native valve; and the lattice structure is configured to remain adhered to leaflets of the native valve, subsequent to exposure of the second component by the de-encapsulation device.

Example 214. The system according to any one of examples 206-207, wherein the delivery structure includes a cavity accommodating the mixture.

Example 215. The system according to any one of examples 206-214, wherein the delivery structure comprises a balloon.

Example 216. The system according to any one of examples 206-215, wherein the delivery structure comprises an implant adapted to be implanted at the anatomical site such that the delivery structure remains within the subject subsequently to exposure of the second component to the first component.

Example 217. The system according to any one of examples 206-215, wherein the delivery structure is absorbed into the subject subsequently to exposure of the second component to the first component.

Example 218. The system according to any one of examples 206-215, wherein the delivery structure is adapted to be removed from the subject subsequently to exposure of the second component to the first component.

Example 219. The system according to any one of examples 178-203, wherein the delivery tool comprises a tool for applying the mixture to skin of the subject.

Example 220. The system according to any one of examples 178-203, wherein the delivery tool comprises a tool for surgically delivering the mixture to the anatomical site.

Example 221. The system according to any one of examples 178-203, wherein the delivery tool comprises an injection tool for injecting the mixture into the subject.

Example 222. The system according to any one of examples 178-203, wherein the delivery tool comprises a subcutaneous delivery tool.

Example 223. The system according to any one of examples 178-222, wherein the de-encapsulation device comprises a chemical adapted, upon introduction into the mixture, to carry out a chemical reaction resulting in de-encapsulating of the second component.

Example 224. The system according to any one of examples 178-222, wherein the de-encapsulation device comprises an energy applicator adapted to apply energy to the mixture.

Example 225. The system according to example 224, wherein the energy applicator comprises an electrode adapted to apply electrical energy to the mixture.

Example 226. The system according to example 224, wherein the energy applicator comprises an electromagnetic radiation source adapted to apply electromagnetic energy to the mixture.

Example 227. The system according to example 224, wherein the energy applicator comprises an ultrasound transducer.

Example 228. The system according to example 227, wherein the ultrasound transducer is adapted to apply high frequency ultrasound energy.

Example 229. The system according to example 227, wherein the ultrasound transducer is adapted to apply high-intensity focused ultrasound (HIFU) energy.

Example 230. The system according to any one of examples 227-229, wherein the ultrasound transducer is an intracorporeal ultrasound transducer.

Example 231. The system according to any one of examples 227-229, wherein the ultrasound transducer is an extracorporeal ultrasound transducer.

Example 232. The system according to any one of examples 227-231, wherein the first component of the mixture is also encapsulated, and wherein the ultrasound transducer is adapted to apply ultrasound energy at a single frequency to de-encapsulate the first component and the second component.

Example 233. The system according to any one of examples 227-231, wherein the first component is also encapsulated, and wherein the ultrasound transducer is adapted to apply ultrasound energy at a first frequency to de-encapsulate the first component and at a second frequency to de-encapsulate the second component.

Example 234. The system according to any one of examples 224-233, wherein a first subset of the capsules includes capsules having a first degree of strength, and a second subset of the capsules comprises capsules having a second degree of strength, and the de-encapsulation device is adapted to apply: a first energy dose to the mixture to de-encapsulate the second component from capsules of the first subset, and a second energy dose to the mixture to de-encapsulate the second component from capsules of the second subset, the first and second energy doses being distinct.

Example 235. The system according to any one of examples 178-234, further comprising an imaging system, adapted to image the mixture at the anatomical site.

Example 236. The system according to example 235, wherein the imaging system comprises an ultrasound transducer adapted to apply low-frequency ultrasound.

Example 237. The system according to example 236, wherein the de-encapsulation device and the imaging system comprise a single ultrasound transducer, adapted to apply high-frequency ultrasound to the mixture for de-encapsulation of the second component, and to apply low frequency ultrasound to the mixture for imaging thereof.

Example 238. The system according to any one of examples 178-237, further comprising a curing energy applicator, adapted to apply energy to the mixture for curing thereof subsequently to exposure of the second component to the first component.

Example 239. The system according to example 238, wherein the curing energy applicator comprises an ultraviolet light source adapted to apply to the mixture electromagnetic energy in the ultraviolet region.

Example 240. The system according to example 238, wherein the curing energy applicator comprises an infrared light source adapted to apply to the mixture electromagnetic energy in the infrared region.

Example 241. The system according to example 238, wherein the curing energy applicator comprises a heating element adapted to heat the mixture.

Example 242. The system according to example 238, wherein the curing energy applicator comprises an electrode adapted to apply electrical energy to the mixture.

Example 243. A system for treating a real or simulated subject, the system comprising a composition comprising a mixture that includes: a first component, and a second component, encapsulated in a manner that prevents its interaction with the first component.

Example 244. The system according to example 243, wherein the mixture is sterile.

Example 245. The system according to any one of examples 243-244, wherein the composition is an adhesive.

Example 246. The system according to any one of examples 243-244, wherein the composition is configured such that interaction of the second component with the first component forms an adhesive.

Example 247. The system according to any one of examples 243-244, wherein the first component is a fluid medium, having suspended therein the encapsulated second component.

Example 248. The system according to any one of examples 243-244, further comprising a suspension medium in which the first component and the encapsulated second component are suspended.

Example 249. The system according to any one of examples 243-248, wherein the composition is configured such that interaction of the second component with the first component hardens the composition.

Example 250. The system according to any one of examples 243-249: further comprising an implant for implantation in the subject, and wherein the composition is held by the implant.

Example 251. The system according to example 250, wherein the composition is held inside the implant.

Example 252. The system according to example 250, wherein the composition is held on the implant.

Example 253. The system according to any one of examples 243-249, further comprising a medical repair structure, formed by de-encapsulation of the second component to facilitate interaction between the first component and the second component.

Example 254. The system according to example 253, wherein the medical repair structure comprises an implant.

Example 255. The system according to example 253, wherein the medical repair structure is adapted for anchoring an implant at an anatomical site of a real or simulated subject.

Example 256. The system according to example 253, wherein the medical repair structure is adapted for augmenting anchoring of an implant at an anatomical site of a real or simulated subject.

Example 257. The system according to example 253, wherein the medical repair structure is adapted for reshaping tissue at an anatomical site.

Example 258. The system according to example 253, wherein the medical repair structure is adapted for adhesion of, or to, tissue at an anatomical site.

Example 259. The system according to example 253, wherein the medical repair structure is adapted for adhesion of an implant at an anatomical site.

Example 260. The system according to example 253, wherein the medical repair structure is adapted to inflate an implant at an anatomical site.

Example 261. The system according to example 253, wherein the medical repair structure forms a coaptation device adapted to improve coaptation of leaflets of a real or simulated heart valve of a real or simulated subject.

Example 262. The system according to example 253, wherein the medical repair structure comprises a spacer adapted to improve function of a real or simulated heart valve of the subject.

Example 263. The system according to any one of examples 243-262, wherein the composition is a liquid.

Example 264. The system according to any one of examples 243-262, wherein the composition is viscoplastic.

Example 265. The system according to any one of examples 243-262, wherein the composition is viscoelastic.

Example 266. The system according to any one of examples 243-262, wherein the composition is a powder.

Example 267. The system according to any one of examples 243-262, wherein the composition is a paste.

Example 268. The system according to any one of examples 243-262, wherein the composition is a gel.

Example 269. The system according to any one of examples 243-262, wherein the composition is a hydrogel.

Example 270. The system according to any one of examples 243-269, wherein the first component is encapsulated in first capsules, and the second component is encapsulated in second capsules.

Example 271. The system according to example 270, wherein the composition further comprises a suspension medium in which both the first capsules and the second capsules are suspended.

Example 272. The system according to example 270, wherein the composition further comprises a powder including the first capsules and the second capsules.

Example 273. The system according to any one of examples 270-272, wherein the first capsules are microcapsules.

Example 274. The system according to any one of examples 270-272, wherein the second capsules are microcapsules.

Example 275. The system according to any one of examples 243-274, wherein the composition further comprises a third component.

Example 276. The system according to example 275, wherein the third component comprises a reinforcing component adapted to mechanically reinforce the composition, when it is hardened.

Example 277. The system according to any one of examples 275-276, wherein the third component comprises an indicator component adapted to enable visualizing of the composition within the subject.

Example 278. The system according to any one of examples 275-277, wherein the third component comprises a surfactant.

Example 279. The system according to any one of examples 275-278, wherein the third component comprises a medicament.

Example 280. The system according to example 279, wherein the medicament is an anti-inflammatory medicament.

Example 281. The system according to any one of examples 279-280, wherein the medicament is an antimicrobial medicament.

Example 282. The system according to any one of examples 243-281, wherein the second component is adapted to be de-encapsulated by application of energy to the composition.

Example 283. The system according to example 282, wherein the second component is adapted to be de-encapsulated by application of high-frequency ultrasound energy to the mixture.

Example 284. The system according to example 282, wherein the second component is adapted to be de-encapsulated by application of high-intensity focused ultrasound (HIFU) energy to the mixture.

Example 285. The system according to example 282, wherein the second component is adapted to be de-encapsulated by application of electrical energy to the mixture.

Example 286. The system according to example 282, wherein the second component is adapted to be de-encapsulated by application of electromagnetic energy to the mixture.

Example 287. The system according to any one of examples 243-281, wherein the second component is adapted to be de-encapsulated by introduction of a chemical agent into the mixture, to carry out a chemical reaction resulting in de-encapsulating of the second component.

Example 288. The system according to any one of examples 270-287, wherein the second capsules comprise polymer capsules.

Example 289. The system according to any one of examples 270-287, wherein the second capsules encapsulating the second component comprise protein capsules.

Example 290. The system according to any one of examples 270-287, wherein the second capsules encapsulating the second component comprise lipid bi-layer capsules.

Example 291. The system according to any one of examples 243-290, wherein: the second component is configured to interact with the first component upon the second component being de-encapsulated, and the composition is curable, subsequently to de-encapsulating of the second component, to form a repair structure.

Example 292. The system according to example 291, wherein the composition is curable by application of energy thereto.

Example 293. The system according to example 292, wherein the composition is curable by application of ultraviolet light thereto.

Example 294. The system according to example 292, wherein the composition is curable by application of infrared light thereto.

Example 295. The system according to example 292, wherein the composition is curable by application of heat thereto.

Example 296. The system according to any one of examples 243-295, wherein the first component and the second component are two components of a two-part foam.

Example 297. The system according to any one of examples 243-295, wherein the first component and the second component are two components of a two-part polyurethane adhesive.

Example 298. The system according to any one of examples 243-295, wherein the first component and the second component are two components of a two-part resin.

Example 299. The system according to example 298, wherein the first component and the second component are two components of a two-part epoxy resin.

Example 300. A system, comprising:
a medical implant, configured to be implanted in a real or simulated subject; and a composition, held by the medical implant, the composition comprising a mixture that includes:
a first component, and a second component, encapsulated in capsules in a manner that prevents its interaction with the first component.

Example 301. The system according to example 300, wherein at least one of the medical implant and the composition is sterile.

Example 302. The system according to any one of examples 300-301, wherein the composition is held inside the implant.

Example 303. The system according to any one of examples 300-301, wherein the composition is held on the implant.

Example 304. The system according to any one of examples 300-303, wherein the composition is an adhesive.

Example 305. The system according to any one of examples 300-303, wherein the composition is configured such that interaction of the second component with the first component forms an adhesive.

Example 306. The system according to any one of examples 300-303, wherein the composition is configured such that interaction of the second component with the first component hardens the composition.

Example 307. The system according to any one of examples 300-306, wherein the composition is a liquid.

Example 308. The system according to any one of examples 300-306, wherein the composition is viscoplastic.

Example 309. The system according to any one of examples 300-306, wherein the composition is viscoelastic.

Example 310. The system according to any one of examples 300-306, wherein the composition is a powder.

Example 311. The system according to any one of examples 300-306, wherein the composition is a paste.

Example 312. The system according to any one of examples 300-306, wherein the composition is a gel.

Example 313. The system according to any one of examples 300-306, wherein the composition is a hydrogel.

Example 314. The system according to any one of examples 300-303, wherein the first component is a fluid medium, having suspended therein the encapsulated second component.

Example 315. The system according to any one of examples 300-303, wherein the composition further comprises a suspension medium in which the first component and the capsules encapsulating the second component are suspended.

Example 316. The system according to any one of examples 300-313, wherein the first component is encapsulated in first capsules, and the capsules encapsulating the second component are second capsules.

Example 317. The system according to example 316, wherein the composition further comprises a suspension medium in which both the first capsules and the second capsules are suspended.

Example 318. The system according to example 316, wherein the composition further comprises a powder including the first capsules and the second capsules.

Example 319. The system according to any one of examples 316-318, wherein the first capsules and/or the second capsules are microcapsules.

Example 320. The system according to any one of examples 300-319, wherein the composition further comprises a third component.

Example 321. The system according to example 320, wherein the third component comprises a reinforcing component adapted to mechanically reinforce the composition, when it is hardened.

Example 322. The system according to example 320, wherein the third component comprises an indicator component adapted to enable visualizing of the composition within the subject.

Example 323. The system according to example 320, wherein the third component comprises a surfactant.

Example 324. The system according to example 320, wherein the third component comprises a medicament.

Example 325. The system according to example 324, wherein the medicament is an anti-inflammatory medicament.

Example 326. The system according to any one of examples 324-325, the medicament is an antimicrobial medicament.

Example 327. The system according to any one of examples 300-326, wherein the second component is adapted to be de-encapsulated by application of energy to the mixture.

Example 328. The system according to example 327, wherein the second component is adapted to be de-encapsulated by application of high-frequency ultrasound energy to the mixture.

Example 329. The system according to example 327, wherein the second component is adapted to be de-encapsulated by application of high-intensity focused ultrasound (HIFU) energy to the mixture.

Example 330. The system according to example 327, wherein the second component is adapted to be de-encapsulated by application of electrical energy to the mixture.

Example 331. The system according to example 327, wherein the second component is adapted to be de-encapsulated by application of electromagnetic energy to the mixture.

Example 332. The system according to any one of examples 300-326, wherein the second component is adapted to be de-encapsulated by introduction of a chemical agent into the mixture, to carry out a chemical reaction resulting in de-encapsulating of the second component.

Example 333. The system according to any one of examples 300-332, wherein the capsules encapsulating the second component comprise polymer capsules.

Example 334. The system according to any one of examples 300-332, wherein the capsules encapsulating the second component comprise protein capsules.

Example 335. The system according to any one of examples 300-332, wherein the capsules encapsulating the second component comprise lipid bi-layer capsules.

Example 336. The system according to any one of examples 300-335, wherein the composition is curable, subsequently to de-encapsulating of the second component, to form a repair structure.

Example 337. The system according to example 336, wherein the composition is curable by application of energy thereto.

Example 338. The system according to example 337, wherein the composition is curable by application of ultraviolet light thereto.

Example 339. The system according to example 337, wherein the composition is curable by application of infrared light thereto.

Example 340. The system according to example 337, wherein the composition is curable by application of heat thereto.

Example 341. The system according to any one of examples 300-340, wherein the first component and the second component are two components of a two-part foam.

Example 342. The system according to any one of examples 300-340, wherein the first component and the second component are two components of a two-part polyurethane adhesive.

Example 343. The system according to any one of examples 300-340, wherein the first component and the second component are two components of a two-part resin.

Example 344. The system according to example 343, wherein the first component and the second component are two components of a two-part epoxy resin.

Example 345. The system according to any one of examples 300-344, wherein the medical implant comprises a lattice structure holding the mixture.

Example 346. The system according to example 345, wherein the lattice structure is configured to remain implanted in the subject, subsequently to interaction of the second component with the first component.

Example 347. The system according to example 345, wherein the system further comprises: a grasper, configured to be advanced transluminally to an anatomical site of the subject, and to hold the lattice structure in contact with tissue of the subject at the anatomical site; and a de-encapsulation device configured to expose the second component to the first component by de-encapsulating the second component while the grasper holds the lattice structure in contact with the tissue at the anatomical site.

Example 348. The system according to example 347, wherein the grasper is configured to be transluminally withdrawn from the subject, subsequently to exposure of the second component by the de-encapsulation device.

Example 349. The system according to example 347, configured such that de-encapsulating the second component while the grasper holds the lattice structure in contact with tissue of the anatomical site adheres the lattice structure to tissue at the anatomical site.

Example 350. The system according to example 349, wherein: the anatomical site is at a native valve of a real or simulated heart of the subject; the grasper is configured to hold the lattice structure in contact with leaflets of the native valve; and the lattice structure is configured to remain adhered to leaflets of the native valve, subsequent to exposure of the second component by the de-encapsulation device.

Example 351. A system at an anatomical site of a real or simulated subject, the system comprising: an implant, comprising: a body, and a composition, held by the body, the composition comprising a mixture that includes:

a first component, and a second component, encapsulated in capsules in a manner that prevents its interaction with the first component; and a delivery tool, configured to deliver the implant to the anatomical site, and comprising a grasper that is configured to hold the body in contact with tissue at the anatomical site.

Example 352. The system according to example 351, wherein at least one of the implant and the delivery tool is sterile.

Example 353. A system at an anatomical site of a real or simulated subject, the system comprising an implant, comprising: a porous matrix, and a composition, disposed on the matrix, the composition comprising a mixture that includes (i) a first component, and (ii) a second component, encapsulated in capsules in a manner that prevents its interaction with the first component.

Example 354. The system according to example 353, wherein at least one of the porous matrix and the composition is sterile.

It should be understood that the use of “and/or” is defined inclusively such that the term “a and/or b” should be read to include the sets: “a and b,” “a or b,” “a,” “b.”

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.). Furthermore, the scope of the present disclosure includes, in some implementations, sterilizing one or more of any of the various systems, devices, apparatuses, etc. in this disclosure.

The techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc. When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

What is claimed is:

1. A system for use inside a heart at a native valve of the heart, the system comprising an implant that comprises:
a porous matrix,
a composition, held in association with the porous matrix, the composition comprising a mixture that includes:
a first component,
a second component, encapsulated in capsules in a manner that prevents its interaction with the first component, the capsules being rupturable to expose the second component to the first component responsively to application of energy to the composition; and
wherein the composition is configured to adhere the porous matrix to one or more leaflets of the native valve upon the exposure of the second component to the first component.

2. The system according to claim 1, wherein the capsules comprise polymer capsules, protein capsules, or lipid bi-layer capsules.

3. The system according to claim 1, wherein the first component is a fluid medium, having suspended therein the capsules.

4. The system according to claim 1, wherein the mixture comprises a powder including the first component and the capsules.

5. The system according to claim 1, wherein the first component and the second component are two components of a two-part foam, are two components of a two-part polyurethane adhesive, or are two components of a two-part resin.

6. The system according to claim 1, further comprising:
a delivery tool, configured to transluminally deliver the implant to the native valve, and
a grasper, configured to hold the implant in contact with the one or more leaflets.

7. The system according to any one of claim 1, further comprising a curing energy applicator, adapted to apply energy to the mixture for curing thereof subsequently to interaction of the second component with the first component.

8. A composition for use inside a heart at a native valve of the heart, the composition comprising:
a first component,
a second component, encapsulated in capsules in a manner that prevents its interaction with the first component, the capsules being rupturable to expose the second component to the first component responsively to application of energy to the composition; and
wherein the composition is configured to adhere to one or more leaflets of the native valve upon the exposure of the second component to the first component.

9. The composition according to claim 8, wherein the capsules comprise polymer capsules, protein capsules, or lipid bi-layer capsules.

10. The composition according to claim 8, wherein the first component is a fluid medium, having suspended therein the capsules.

11. The composition according to claim 8, wherein the composition comprises a powder including the first component and the capsules.

12. The composition according to claim 8, wherein the first component and the second component are two components of a two-part foam, are two components of a two-part polyurethane adhesive, or are two components of a two-part resin.

13. A method of adhering leaflets of a native valve inside a heart:
holding a composition in association with a porous matrix;
wherein the composition comprises a mixture that includes:
a first component,
a second component, encapsulated in capsules in a manner that
prevents its interaction with the first component;
rupturing the capsules to expose the second component to the first component; and
adhering the porous matrix to two or more of the leaflets of the native valve with the composition upon the exposure of the second component to the first component.

14. The method according to claim 13, wherein the capsules comprise polymer capsules, protein capsules, or lipid bi-layer capsules.

15. The method according to claim 13, wherein the first component is a fluid medium, having suspended therein the capsules.

16. The method according to claim 13, wherein the mixture comprises a powder including the first component and the capsules.

17. The method according to claim 13, wherein the first component and the second component are two components of a two-part foam, are two components of a two-part polyurethane adhesive, or are two components of a two-part resin.

18. The method according to claim 13, further comprising:
transluminally delivering an implant that contains the composition to the native valve, and
holding the implant in contact with one or more leaflets.

19. The method of claim 18 wherein the implant is held in contact with the one or more leaflets until the composition cures.

20. The method according to claim 13, further comprising applying energy to the mixture for curing thereof subsequently to interaction of the second component with the first component.
